(19) United States
(12) Patent Application Publication
(10) Pub. No.: US 2022/0361776 A1
(43) Pub. Date: Nov. 17, 2022

(54) SENSING DEVICES BASED ON MICRONEEDLE ARRAYS FOR SENSING APPLICATIONS INCLUDING KETONE BODIES MONITORING

(71) Applicants: THE REGENTS OF THE UNIVERSITY OF CALIFORNIA, Oakland, CA (US); President and Fellows of Harvard College, Cambridge, MA (US)

(72) Inventors: Joseph Wang, San Diego, CA (US); Hazhir Teymourian, La Jolla, CA (US); Eyal Dassau, La Jolla, CA (US)

(21) Appl. No.: 17/755,211

(22) PCT Filed: Oct. 23, 2020
(86) PCT No.: PCT/US2020/057229

§ 371 Date: Apr. 22, 2022

Related U.S. Application Data
(60) Provisional application No. 62/925,201, filed on Oct. 23, 2019.

(51) Int. Cl. A61B 5/145; A61B 5/00; A61B 5/1486

(52) U.S. Cl. CPC A61B 5/14503; A61B 5/14532; A61B 5/685; A61B 5/14865; A61B 5/002; A61B 5/14514

(57) ABSTRACT

Disclosed is a wearable microneedle sensor platform for minimally-invasive, real-time monitoring of key biomarkers. In some aspects, a device includes a wearable epidermal sensor comprising an array of hollowed needles, each hollowed needle having a protruded needle structure including multiple layers forming a hollow interior, at least one hollowed needle including a working electrode to interact with one or more chemical or biological substances that come in contact with the protruded needle structure, at least one hollowed needle including a counter electrode to measure an electrical potential difference with the working electrode; and a wireless transmitter in communication with the sensor to generate output signals based on the electrical potential difference with the working electrode. The output signal represents β-hydroxybutyrate as a biomarker of ketone bodies.

### CROSS-REFERENCE TO RELATED APPLICATION

This patent document claims priority to and benefits of U.S. Provisional Appl. No. 62/925,201, titled “SENSING DEVICES BASED ON MICRONEEDLE ARRAYS FOR SENSING APPLICATIONS INCLUDING KETONE BODIES MONITORING” and filed on Oct. 23, 2019. The entire contents of the before-mentioned patent application are incorporated by reference as part of the disclosure of this document.

### TECHNICAL FIELD

This patent document relates to a biosensor capable of continuous and stable detection of biomarkers.

### BACKGROUND

Diabetes mellitus is a metabolic disease associated with high blood sugar due to insufficient production of insulin by the body or inadequate response by cells to the insulin that is produced. Diabetic ketosis or diabetic ketoacidosis is a life-threatening condition that occurs in patients with diabetes mellitus. Detecting altered concentrations of ketones in the blood and urine is crucial for the diagnosis and treatment of diabetic ketosis or diabetic ketoacidosis.

### SUMMARY

Disclosed are systems, methods and devices directed to reagentless and continuous monitoring of diabetes biomarkers, such as ketone bodies, using a microneedle platform with an array of microneedles capable of providing real-time analytical data of ketone bodies' levels, which can be used for detecting diabetic ketosis (DK) and/or diabetic ketoacidosis (DKA) in diabetes patients.

The technology disclosed in this patent document can be implemented in some embodiments to create a wearable minimally-invasive system for continuous monitoring of the DK or DKA based on the simultaneous minimally-invasive monitoring of glucose and beta-hydroxybutyrate in the interstitial fluid.

In some embodiments in accordance with the present technology, an epidermal electrochemical sensor device includes an electrochemical sensor comprising a substrate and an array of microneedles disposed on the substrate, each microneedle having a protruded needle structure and one of a hollow interior inside the protruded needle structure or a coating over at least a portion of the protruded needle structure, wherein the array of microneedles comprises at least one microneedle that includes a working electrode configured within the hollow interior or in the coating to interact with one or more chemical or biological substances that come in contact with the protruded needle structure, and at least one microneedle including a counter electrode configured within the hollow interior or in the coating to measure an electrical potential difference with the working electrode; and a transmitter in communication with the electrochemical sensor to generate output signals based on the electrical potential difference with the working electrode and the counter electrode.

In some embodiments in accordance with the present technology, an epidermal electrochemical sensor device for detection of diabetes biomarkers includes a substrate; a plurality of microneedle electrodes coupled to the substrate and operable to penetrate within skin and contact the microneedle electrodes with interstitial fluid when the device is attached to skin of a user, each microneedle electrode including a microneedle structure and an electrode structure, wherein each microneedle structure includes an exterior wall spanning outward from a base surface of the microneedle structure and forming an apex at a terminus point of the exterior wall, and the electrode structure is configured within a hollow interior region of the microneedle structure or on at least a portion of the exterior wall of the microneedle structure; an enzymatic functionalization layer coupled to the electrode structure of the first microneedle electrode of the plurality of microneedle electrodes operable to detect β-hydroxybutyrate (HB) in the interstitial fluid through an electrochemically-mediated enzymatic reaction, wherein the enzymatic functionalization layer is immobilized to the electrode structure and comprises a β-hydroxybutyrate dehydrogenase (HBD) enzyme and HBD-cofactor that is unbound to the HBD enzyme; and a redox mediator coupled to the electrode structure of the first microneedle electrode to facilitate electron transfer in the electrochemically-mediated enzymatic reaction, wherein the plurality of microneedle electrodes includes a counter electrode or a reference electrode, or both, configured to apply or detect an electrical signal between the counter electrode and/or reference electrode and the first microneedle electrode.

In some embodiments in accordance with the present technology, a sensor device includes a plurality of protruded working needle structures including a first working electrode structured to perform a first electrochemical detection of a first chemical or biological substance and a second working electrode structured to perform a second electrochemical detection of a second chemical or biological substance; and a plurality of protruded functional needle structures including a reference electrode structured to provide a reference electrical potential to the plurality of protruded working needle structures and a counter electrode structured to measure an electrical potential difference between the reference electrical potential and the protruded working needle structures, wherein the first and second working electrodes are structured to share the counter electrode and the reference electrode to detect the first and second chemical or biological substance simultaneously.

In some embodiment of the disclosed technology, a sensor device includes an array of hollowed needles, each hollowed needle having a protruded needle structure including multiple layers forming a hollow interior, at least one hollowed needle including a working electrode to interact with one or more chemical or biological substances that come in contact with the protruded needle structure, at least one hollowed needle including a counter electrode to measure an electrical potential difference with the working electrode, and a transmitter in communication with the array of hollowed needles to generate output signals based on the electrical potential difference with the working electrode. The output signal represents β-hydroxybutyrate as a biomarker of ketone bodies.

In some embodiment of the disclosed technology, a sensor device includes an array of microneedle sensors including a plurality of protruded needle structures each including a hollow interior and a plurality of electrodes arranged in the hollow interiors of the plurality of protruded needle structure, respectively, wherein the plurality of electrodes includes a first working electrode structured to interact with a first chemical or biological substance and a second working electrode structured to interact with a second chemical or biological substance, and wherein the first and second working electrodes are configured to detect the first and second chemical or biological substance simultaneously, and a transmitter in communication with the array of microneedle sensors to generate output signals corresponding to the first and second chemical or biological substance.

The subject matter described in this patent document can be implemented in specific ways that provide one or more of the following features.

### BRIEF DESCRIPTION OF THE DRAWINGS

FIG. 1A shows a diagram illustrating an example embodiment of an epidermal electrochemical sensor platform in accordance with the disclosed technology, including an array of hollow microneedles capable of providing real-time analytical data of biomarkers in interstitial fluid, such as ketone bodies' levels to monitor diabetic ketosis and/or diabetic ketoacidosis in diabetes patients.

FIGS. 1B-1D show diagrams illustrating an example of dual-marker β-hydroxybutyrate (HB)/glucose (GL) sensing on a microneedle sensor platform in accordance with some embodiments of the disclosed technology.

FIG. 1E shows a diagram illustrating another example embodiment of an epidermal electrochemical sensor platform in accordance with the disclosed technology, including an array of solid microneedles capable of providing real-time analytical data of biomarkers in interstitial fluid, such as ketone bodies' levels to monitor diabetic ketosis and/or diabetic ketoacidosis in diabetes patients.

FIGS. 2A-2D show diagrams and plots showing nicotinamide adenine dinucleotide, reduced form (NADH) electrocatalysis analysis on an example ionic liquid (IL)-based carbon paste microneedle sensor platform in accordance with some embodiments of the disclosed technology.

FIGS. 3A-3F show data plots depicting a performance evaluation of an example HB microneedle sensor.

FIGS. 4A-4C show cross-talk study towards simultaneous HB/GL dual-analyte measurements.

FIG. 5 shows an example schematic of microneedle-based electrochemical biosensor for real-time monitoring of dual glucose/HB markers in interstitial fluid (ISF).

FIGS. 6A-6C show examples of microneedle-based electrochemical biosensor based on some implementations of the disclosed technology.

FIG. 7 shows an example of sensor patch that shows electronic interface and integration implemented based on some embodiments of the disclosed technology.

FIG. 8 shows an example of system integration and a microneedle-based electrochemical biosensor board interface.

### DETAILED DESCRIPTION

Disclosed are methods, systems, and devices that pertain to reagentless and continuous ketone bodies monitoring (CKM) using a microneedle sensor platform with an array of microneedles capable of providing real-time analytical data of ketone bodies' levels to monitor diabetic ketosis (DK) and/or diabetic ketoacidosis (DKA) in diabetes patients.

Some embodiments of the disclosed CKM microneedle sensor technology include a device that can be easily worn on the individual's skin and measure the concentration of β-hydroxybutyrate (HB), an important index of ketone bodies, directly inside the interstitial fluid (ISF) of the skin. For example, in some embodiments, the analyte detection and HB level analysis is performed through electrochemical enzymatic detection of HB on the surface of functionalized microneedle electrodes (e.g., carbon paste-packed hollow microneedle electrodes) and engineered to facilitate β-hydroxybutyrate dehydrogenase (HBD) enzyme-catalyzed oxidation of HB to acetylacetate (AcAc) with the concomitant reduction of cofactor NAD+ (nicotinamide adenine dinucleotide, oxidized form) to NADH (nicotinamide adenine dinucleotide, reduced form). The disclosed technology effectively integrates this electrochemical enzymatic detection scheme into a portable, epidermal sensor device using a microneedle area to penetrate into a surface layer of the skin to contact the ISF and direct and control the redox reaction while simultaneously recording the resultant electric signals continuously and in real-time, thereby allowing fast on-the-spot detection of one or multiple key diabetic biomarkers, such as ketone bodies (e.g., via HB) and/or glucose.

Diabetic ketoacidosis, a severe complication of diabetes mellitus with potentially fatal consequences, is characterized by hyperglycemia and metabolic acidosis due to the accumulation of ketone bodies. Due to its rapidity of potential onset and life-threatening aspects, diabetic ketoacidosis requires diabetes patients to be constantly aware of both glucose and ketone bodies. Yet, there are few if any viable technologies that allow diabetic patients the ability to even monitor both analytes (glucose and ketone bodies) in a concurrent, consistent, and minimally-invasive or obtrusive way. Despite major advances in diabetes management mainly since the emergence of new-generation continuous glucose monitoring (CGM) devices capable of in-vivo monitoring of glucose directly in the interstitial fluid, the continuous monitoring of ketone bodies needs further attention and improvement to arrive at an effective solution for diabetic patients to monitor all important diabetes biomarkers for managing their diabetes.

The disclosed technology can be implemented, in some embodiments, to provide a real-time CKM microneedle platform based on the electrochemical monitoring of β-hydroxybutyrate, as a dominant biomarker of ketone bodies. Such a real-time assay, based on the β-hydroxybutyrate dehydrogenase enzyme biocatalytic reaction, is realized by addressing the major challenges associated with the stable confinement of the enzyme/cofactor couple (HBD/NAD+) and by realizing a low-potential selective and fouling-free NADH oxidation. The CKM microneedle device provides an attractive analytical performance, with high sensitivity (e.g., with low detection limit, 50 μM), high selectivity in the presence of potential interferences, along with good stability during prolonged operation in artificial ISF. Example implementations of various embodiments of the CKM device demonstrate the applicability of this microneedle sensor array toward wearable non-invasive on-body applications, including experiments demonstrating the CKM technology in phantom gel skin-mimicking model, and the integration of the CKM detection of HB with the glucose (GL) detection on a single microneedle array toward real-time dual-analyte HB/GL detection. Such microneedle sensor array can be utilized towards the simultaneous monitoring of additional diabetes-related biomarkers toward a tight glycemic control.

DKA is a life threatening complication of both type 1 and type 2 diabetes mellitus and continues to have high rates of morbidity and mortality despite advances in the treatment of diabetes. DKA is caused by an effective lack of insulin, usually accompanied by an increase in counter-regulatory hormones such as glucagon, cortisol and epinephrine. This hormonal imbalance leads to severe hyperglycemia and production of large quantities of ketone bodies as a side product of fat breakdown and consequent metabolic acidosis. In this regard, the ability to monitor rising levels of both glucose and ketone bodies is of paramount importance to prevent, diagnose and treat the DKA. It has also been shown that before raising the glucose concentration, the ketones level increases apparently after the interruption of insulin administration; thus, measuring ketone bodies can represent a useful mean for early diagnosis of insulin deprivation. However, current approaches to continuously monitor the diabetic patients rely solely on the glucose measurements by CGM devices, which provide real-time data of glucose levels in the ISF. Ketone bodies are commonly tested in-vitro by using capillary blood meters which measure β-hydroxybutyrate (HB), as the most important index in DKA diagnosis. While such capillary test strips enable rapid assessment of blood HB levels, they require pricking the fingertip for periodic measurements and cannot track the trends and real-time fluctuations of HB concentrations.

The device implemented based on some embodiments of the disclosed technology can perform continuous ketone bodies monitoring (CKM) along with glucose, providing rapid diagnosis and treatment of the diabetic ketosis DK (a transient condition characterized by elevated levels of ketone bodies) and diabetic ketoacidosis (DKA) for diabetic patients.

ISF has recently shown considerable promise for diagnostic and monitoring purposes, as a strong correlation has been demonstrated between the population of small molecules, electrolytes, and proteins in the ISF with that of plasma. In this regard, microneedle sensors have garnered much attention over the last decade due to their ability to access the dermal ISF without causing pain or bleeding, while gathering a wealth of molecular information concerning disease markers and metabolites. In case of diabetes management, while microneedle-based ISF glucose sensing is actively pursued as a minimally-invasive alternative to the current CGM devices, there have been no efforts towards the continuous monitoring of ketone bodies in ISF.

The disclosed technology can be implemented in some embodiments to provide a CKM microneedle sensor array structured to perform real-time continuous monitoring of HB, alongside with glucose detection. The disclosed microneedle-based CKM and CKM/GL monitoring technology addresses and overcomes key challenges related to the specific bioelectrocatalytic processes involved in the electrochemical detection of HB detection based on the NAD-dependent dehydrogenase enzyme. Unlike common oxygen-dependent oxidase enzyme electrodes, dehydrogenase-based electrochemical biosensors require the stable surface confinement of the NAD+ cofactor. Besides the challenge of stable NAD+ immobilization, such amperometric biosensors require high overvoltage for the anodic detection of the NADH reaction product. Such high NADH detection potential results in severe electrode surface fouling and is coupled to interferences from coexisting electroactive constituents. As a result, unlike common oxygen-based monitoring platforms, dehydrogenase enzymes have rarely been used for continuous monitoring applications. The aforementioned issues may be addressed through a synergistic combination of judiciously chosen biosensor components based on some embodiments of the disclosed technology.

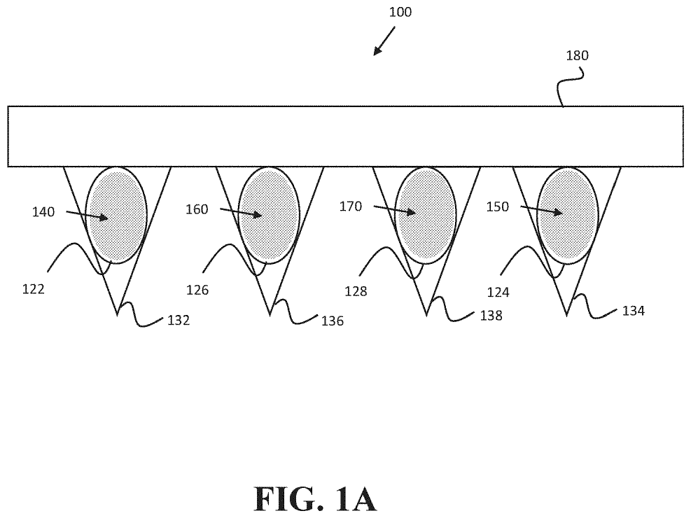

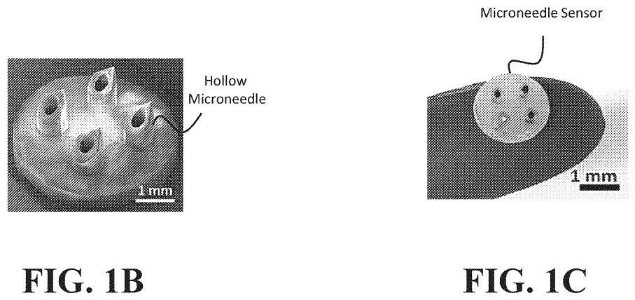

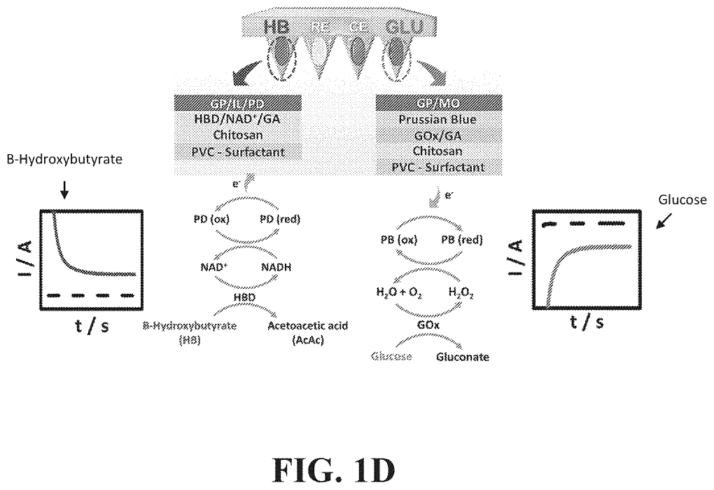

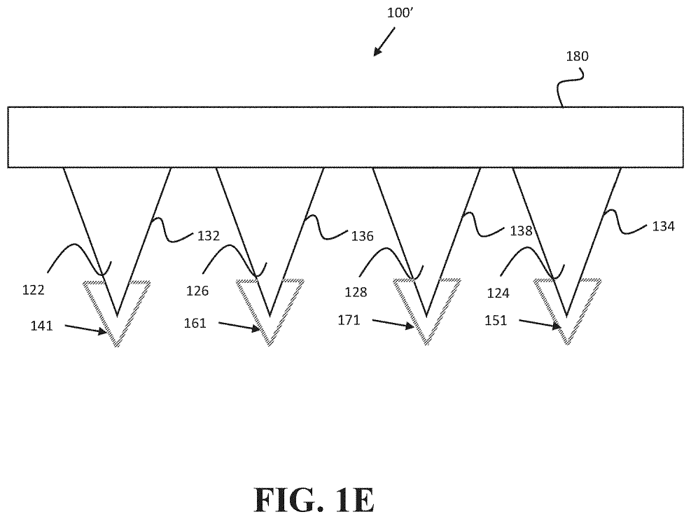

In some embodiments of a reagentless and continuous monitoring epidermal electrochemical sensor device, for example, a functional multilayer design may be adopted to construct the sensing array, relying on an ionic liquid (IL)-based carbon paste (CP) transducer electrode incorporated with the phenanthroline dione (PD) acting as the mediator, followed by HBD/NAD+ mixture immobilization, glutaraldehyde (GA) crosslinking and coating with chitosan (Chit) and polyvinylchloride (PVC) as outer polymer layers, as will be discussed below with reference to FIGS. 1A-1D. The used IL, with imidazolium-based cation and trifluoromethyl sulfonylimide anionic groups, plays a dual function; (i) stabilizing and thus confining the biomolecules, including small-sized NAD+, through hydrogen bonding (between the fluoride/sulfonyl groups of ionic liquid (IL) and hydroxyl/amine related hydrogen atoms of NAD+) along with Coulombic interactions (mostly between phosphate groups of NAD+ and imidazolium-based cation); (ii) imparting fouling-resistant and high sensitivity to the biosensor toward NADH detection. Such stabilizing and fouling-resistance effects of ILs have been reported. Adsorption inside the pores of the underlying electrode, crosslinking via GA to form an extended 3D network of HBD/NAD+ and the outer polymer layers of Chit and PVC also contributed toward constraining of biomolecules. The PD mediator offered a low-potential, interference-free NADH electrocatalysis, a high compatibility with HBD enzyme along with negligible solubility and leaching. Finally, the combination of semipermeable Chit and permselective PVC polymeric layers provided effective anti-biofouling properties toward practical scenarios in physiological body fluids. Addition of Triton x-100 as a non-ionic surfactant into the PVC may further improve biosensor's resistance against biofouling and broadened its detection range. Such HBD-based microneedle sensor may result in a highly sensitive, selective and stable amperometric measurements of the target β-hydroxybutyrate marker, as demonstrated for prolonged operation in artificial ISF and using a phantom gel skin-mimicking model. The resulting HB microneedle sensor may be integrated with an oxidase-based glucose microneedle sensor on the same array platform, leading to an attractive electrochemical HB/GL sensor array towards the simultaneous real-time continuous ISF monitoring of these diabetes markers. This dual-analyte amperometric sensor may be realized through packing of appropriate electrode materials within the hollow microneedle tips where the simultaneous HB/GL detection takes place. The attractive analytical performance of the new microneedle sensor array holds great promise toward continuous real-time noninvasive assessment of DK/DKA for diabetic patients, towards the development of a feedback control predictive algorithms for automated diabetes management.

FIGS. 1A-1D show diagrams of example embodiment of an epidermal electrochemical sensor device for real-time monitoring of diabetic biomarkers in interstitial fluid, using an array of specially-functionalized microneedle electrodes capable of reagentless detection of ketone bodies' levels to monitor diabetic ketosis and/or diabetic ketoacidosis in diabetes patients. FIG. 1A shows a diagram of an example microneedle electrode array of the sensor platform, and FIGS. 1B-1D shows images, illustrations, and data plots depicting example embodiments and implementations of dual-marker β-hydroxybutyrate (HB)/glucose (GL) sensing on the microneedle sensor platform, like that in FIG. 1A. Specifically, FIG. 1B shows a scanning electron microscope (SEM) image of the computer numerical control (CNC)-fabricated MN showing 2×2 array of hollow microneedles. FIG. 1C shows the optical image of the packed MN sensor, showing working electrode (WE) for HB (β-hydroxybutyrate) packed with graphite powder (GP)/ionic liquid (IL)/phenanthroline dione (PD), a WE for GL filled with graphite powder (GP)/mineral oil (MO) paste and another GP/MO packed microneedle acting as counter electrode (CE). An Ag/AgCl wire (500 μm)-integrated microneedle acts as a reference electrode (RE). FIG. 1D shows the schematic illustration of the dual-analyte amperometric detection mechanism on multilayer modified sensors for HB (left) and GL (right). Typical corresponding amperograms obtained upon HB and GL detection are shown for HB (left) and GL (right).

Referring to FIG. 1A, the sensor platform includes a unit array 100 of microneedles. In some embodiments, the unit array 100 of microneedles includes a substrate 180, and a plurality of microneedles 132, 134, 136, 138, each including a hollow region (122, 124, 126, 128) inside a microneedle structure of the microneedles 132, 134, 136, 138, formed on the substrate 180. The microneedles 132, 134, 136, 138 of the sensor array 100 include a microneedle structure and an electrode structure. The microneedle structure can include an exterior wall spanning outward from a base surface (e.g., the base located at the substrate 180 surface), which forms an apex at a terminus point of the exterior wall. The microneedle structure includes, at a portion of the exterior wall, an opening leading in to the hollow region (or cavity) of the needle structure, which includes an interior wall. In some embodiments, the microneedle structure can be configured in a pyramidal geometry (e.g., trigonal pyramidal with three exterior walls or square pyramidal with four exterior walls); whereas in some embodiments, the microneedle structure can be configured in a conical geometry (e.g., one curved, tapered exterior wall).

The electrode structure can be contained within the microneedle structure. In some embodiments, for example, the electrode structure includes an electrically conductive material that is embedded, at least partially, within the hollow interior of the microneedle structure. In some embodiments, for example, the hollow region in each of the microneedle structure contains one or more functionalization materials that are configured to interact with a certain substance for directing and controlling a redox reaction for electrochemical detection of a target analyte. In various embodiments, for example, each microneedle of the unit array 100 can include an electrically-conductive material of the electrode structure (not shown) that is electrically connected with a conduit or via, e.g., which can be on top or within the substrate 180, to transfer electrical signals to an electronic interface of an electrical circuit of the sensor device.

In some implementations, the plurality of microneedles may include first to fourth microneedles 132, 134, 136, 138 including first to fourth hollow regions 122, 124, 126, 128, respectively. In some examples, the first hollow 122 of the first microneedle 132 includes a first material that interacts with a first substance, and the second hollow 124 of the second microneedle 134 includes a second material that interacts with a second substance. In some examples, the first substance may include β-hydroxybutyrate (HB), and the second substance may include glucose (GL).

In some implementations, the first hollow region 122 of the first microneedle 132 includes a working electrode (WE) assembly for HB detection comprising a first material or a first group of materials 140 that interact with β-hydroxybutyrate (HB). The first material or the first group of materials 140 may include at least one of graphite powder (GP), ionic liquid (IL), or phenanthroline dione (PD). For example, the working electrode of the first microneedle 132 can be engineered to detect HB utilizing an ionic liquid-based carbon paste (IL-CP) transducer electrode incorporated with the phenanthroline dione (PD) acting as a mediator for a redox reaction. In some implementations, the first material or the first group of materials 140 of the WE assembly may also include at least one of β-hydroxybutyrate dehydrogenase (HBD), NAD+ (nicotinamide adenine dinucleotide, oxidized form), or glutaraldehyde (GA). The HBD, NAD+, and/or GA can be configured in a layer partially or fully attached to the WE base layer, e.g. the IL-CP-PD electrode structure or layer. In some implementations, these materials may be coated with chitosan (Chit). In some implementations, the first material or the first group of materials 140 may also include polyvinylchloride (PVC) as an outer polymer layer. In one example, the first group of materials 140 in the first hollow may form a multilayer that includes a plurality of material layers discussed above. In another example, the first group of materials 140 in the first hollow 122 may form a mixture including the plurality of material layers discussed above.

In some implementations, the second hollow region 124 of the second microneedle 134 includes a second working electrode (WE) assembly for GL detection comprising a second material or a second group of materials 150 that interact with glucose (GL). In some implementations, the second material or the second group of materials 150 may include at least one of graphite powder (GP) or mineral oil (MO) paste. In some implementations, the second material or the second group of materials 150 may also include Prussian Blue (PB) material. In some implementations, the second material or the second group of materials 150 may also include glucose oxidase (GOx) enzyme or glutaraldehyde (GA). In some implementations, these materials may be coated with chitosan (Chit). In some implementations, the first hollow may also include polyvinylchloride (PVC) as an outer polymer layer. In one example, the second group of materials 150 in the second hollow 124 may form a multilayer that includes a plurality of material layers discussed above. In another example, the first group of materials 140 in the first hollow may form a mixture including the plurality of material layers discussed above.

In some implementations, the third hollow 126 of the third microneedle 136 includes a third material or a third group of materials 160 that are suitable for a counter electrode (CE). In some implementations, the third material or the third group of materials 160 may include at least one of graphite powder (GP) or mineral oil (MO) paste.

In some implementations, the fourth hollow 128 of the fourth microneedle 138 includes a fourth material or a fourth group of materials 170 that are suitable for a reference electrode (RE). In some implementations, the fourth material or the fourth group of materials 170 may include an Ag/AgCl wire.

In various embodiments, the unit array 100 can utilize a common reference electrode and counter electrode for the simultaneous detection of the HB and GL, as illustrated in FIG. 1D, for example.

The disclosed technology can be implemented based on some embodiments to provide a microneedle sensing platform capable of continuous real-time monitoring of HB along with glucose. The microneedles may be fabricated by example methods of highly cost-effective, reproducible (1 μm precision), and scalable CNC-micromachining technique based on some embodiments of the disclosed technology. The SEM image of the CNC-fabricated microneedle array based on some embodiments includes a plurality of microneedles. By way of example, FIGS. 1B-1D illustrate four hollow microneedles (e.g., 500 μm i.d.) that form the two working electrodes (WEs) for HB and GL detection based on the carbon paste (CP) transducers, along with two microneedles acting as CE and RE.

Different surface chemistries and detection mechanisms may be utilized on the CP-filled electrode surfaces to enable the in-situ analyte monitoring. As illustrated in FIG. 1D, the HB analysis is performed through an electrochemical mediated enzymatic approach, relying on the HBD-catalyzed oxidation of HB to acetylacetate (AcAc) with the concomitant reduction of NAD+ (nicotinamide adenine dinucleotide, oxidized form) to NADH (nicotinamide adenine dinucleotide, reduced form). The produced NADH oxidizes back to NAD+ by using PD as the electron shuttling reagent to continuously regenerate the cofactor.

In some embodiments of the disclosed technology, the CKM of the sensor device can be realized through a carefully engineered layer-by-layer modification protocol to make sure the following criteria are met: (1) all the components of the biosensor including mediator, enzyme and cofactor are confined on the microneedle electrode surface; (2) the strategy used for confinement of the components should not affect/limit their electron transfer dynamics (sensitivity); and/or (3) the potential leaching of the components is avoided to provide both a stable electrochemical signal and ensure the safety of the sensing platform for in-vivo human trials. The synergistic combination of various factors can enable highly sensitive and selective HB detection along with a highly stable current response for long-term continuous monitoring. The modification protocol can include appropriate selection of the mediator, e.g., which can be based on the phenanthroline dione chemistry, with very low solubility for fast, low-potential NADH detection and minimized leaching of the mediator. In some implementations, the modification protocol can include an IL-based CP electrode to minimize the surface fouling due to the NADH oxidation products and thus, provide a stable current response at the working electrode. The modification protocol can include immobilization of the HBD/NAD+ mixture through adsorption into the pores of the underlying CP electrode, followed by GA crosslinking which allows a relatively free access of the components to each other and thus, a facilitated, efficient transfer of electrons from HB target analyte to the electrode surface. Also, the modification protocol can include a combination of Chit (Chitosan) and PVC (Polyvinyl chloride) as semipermeable and permselective outer membranes, respectively, in order to reduce the biofouling on the electrode surface.

Additionally or alternatively, for example, GL detection may be built upon the enzymatic GOx (Glucose Oxidase)-catalyzed GL oxidation to gluconate and the reduction of the produced hydrogen peroxide ((H₂O₂)) on the PB (Prussian Blue)-modified MO-based CP electrode (FIGS. 1B-1D). Such an integrated dual-analyte HB/GL sensing system onto a single microneedle platform toward developing a wearable non-invasive patch capable of real-time continuous ISF monitoring in diabetic patients greatly enhances the quality of diabetes management by extracting further analytical information. Furthermore, the utilized design for the continuous ketone bodies monitoring (CKM), addressing the limitations of nicotinamide adenine dinucleotide (NAD)-dependent dehydrogenase-based biosensors, can be readily extended for the continuous analysis of other interstitial fluid (ISF) biomarkers in connection to the related dehydrogenase enzyme.

In some implementations, a highly important aspect of some embodiments of the disclosed technology is that the continuous analysis is based on the use of NAD-dependent dehydrogenase type enzyme (β-hydroxybutyrate dehydrogenase, HBD), which the high abundance of these type of enzymes in biosystems and their advantages compared to other oxireductase enzymes makes the system implemented based on some embodiments of the disclosed technology highly applicable to the continuous monitoring of any other metabolite biomarker in connection with the relevant dehydrogenase enzyme. The system implemented based on some embodiments of the disclosed technology demonstrates a stable electrochemical detection of the NADH reaction product, with no apparent surface fouling along with stable confinement of the NAD+ HBD enzyme cofactor. These facilitate the continuous detection of other important substrates of dehydrogenase enzymes. It has been demonstrated to work for sensitive and selective continuous amperometric detection of physiological concentration ranges of HB in the artificial ISF samples. Some example microneedle CKM biosensor devices, which can utilize NAD-dependent dehydrogenase enzyme, can be combined with the continuous monitoring of a wide variety of other biomarkers such as glucose, alcohol, glutamate, lactate using the corresponding dehydrogenase or oxidase enzymes on the same microneedle array patch. Other important feature of some embodiments of the disclosed technology is the simultaneous detection of HB along with glucose (GL) on a single microneedle sensor array, providing a more comprehensive understanding of the patient's state of health than single marker measurement (FIGS. 1B-1D) toward enhanced glycemic control. The new innovations can be applied to the CKM using other on-body sensing platforms or modalities.

In some embodiments of the disclosed technology, the microneedle CKM system may be used to demonstrate a NAD-dependent dehydrogenase based biosensor capable of continuous and stable detection of biomarkers over prolonged periods of time. The electrochemical platform implemented based on some embodiments of the disclosed technology may be used to non-invasively target the highly important ketone bodies biomarkers in ISF body fluid as an alternative to other body fluids used for ketone bodies measurement, i.e., blood and urine. In some embodiments of the disclosed technology, the sensing platform lies in the possibility of the integration of CKM microneedle with other important biomarkers on a single microneedle array platform as it has been demonstrated for the simultaneous dual-analyte detection of HB and GL. In some embodiments of the disclosed technology, the sensor is easily adoptable to a wearable fully-integrated microneedle patch capable of multiplexed detection of various analytes in a real-time fashion.

The NAD-dependent dehydrogenase enzymes constitute the largest family of known enzymes and relate to a vast number of substrates. They offer several advantages when compared to other oxireductase enzymes such as oxidases which are commonly employed in biosensors fabrication. The most important advantage relates to their oxygen insensitivity which is a serious problem in current commercial biosensors in the market. However, in order to develop a wearable continuous monitoring system based on dehydrogenase enzymes, there are several challenges need to be addressed. First, NAD+ cofactor is not bound to the enzyme and thus, should be immobilized along with the enzyme. This can be a quite challenging task regarding the small size of NAD+ molecule. Second challenge lies in the fact that the oxidation of NADH is usually irreversible, it occurs at high potential, NAD+ can act as an inhibitor of the direct electrode process or even provokes electrode surface poisoning. Thus, the use of a suitable mediator molecule is inevitable in the biosensors and biofuel cells relying on dehydrogenase enzymes. The disclosed technology can be implemented in some embodiments to provide a unique combination of polymeric layers including chitosan and polyvinylchloride (PVC) to efficiently and durably confine the enzyme and cofactor biomolecules onto the carbon packed microneedle electrode surface in an active form while keeping enough mobility to ensure the electron transfers to occur between them. Also, the combination of ionic liquid (IL) and phenantroline dione (PD) mediator ensured the stable, sensitive and low-potential detection of HB analyte to ensure the regeneration of NAD+ through continuous oxidation of produced NADH during the enzymatic reaction. The system implemented based on some embodiments of the disclosed technology demonstrates a stable confinement of the NAD+ cofactor along with a stable electrochemical detection of the NADH reaction product, with no apparent surface fouling, and facilitating the continuous detection of variety of important substrates of dehydrogenase enzymes.

The population of small molecules, electrolytes, and proteins in the interstitial fluid (ISF) surrounding dermal cells strongly correlates with that of blood and thus use of ISF has recently garnered extensive interest as the first choice biofluid for the wearable sensing applications (e.g., in commercial CGM devices). However, ketone bodies detection has never been investigated in the ISF and all the current methods focus on blood or urine for their determination.

In some embodiments of the disclosed technology, the sensor is the multiplexed simultaneous detection of HB along with glucose toward the real-time monitoring of DK/DKA in diabetic patients. The disclosed technology can be implemented in some embodiments to realize such a simultaneous dual analyte HB/GL detection through carefully designed enzymatic approaches for these analytes in order to avoid the potential cross-talk between them. The smart architecture of different surface chemistries employed in the microneedle sensor platform implemented based on some embodiments of the disclosed technology enables a user friendly approach toward the multiplexed wearable combination of continuous glucose monitoring (CGM)/continuous ketone bodies monitoring (CKM).

While the continuous monitoring of HB analyte has been demonstrated in this patent document, the current system can be extended to the analysis of any other analyte based on the dehydrogenase enzyme. Also, the multiplexed concept herein reported for HB/GL can be applied/expanded to cover other biomarkers including, but not limited to, lactate, alcohol, glutamate etc.

Currently there is no available device for the continuous monitoring of ketone bodies and the only methods available for measuring ketone bodies are based on the urine dipsticks or capillary blood meters, with the latter providing higher sensitivity and specificity in identifying the DK/DKA. While capillary meters enable rapid assessment of blood HB levels, however they are invasive as they require pricking the fingertip to acquire the blood sample and more importantly, they cannot track the analyte trends and fluctuations. Also, there is not any literature report targeting the HB analyte in the ISF.

Also, the current blood test strips can only detect a single biochemical marker, i.e., HB or glucose. Obviously, developing a dual-marker sensor capable of providing continuously monitor the dynamically-changing levels of both HB and glucose markers at the same time would be of critical significance for diabetes management and treatment.

FIG. 1E shows a diagram of another example embodiment of the microneedle unit array for an electrochemical sensor platform in accordance with the disclosed technology. For example, in some embodiments, the disclosed reagentless, continuous epidermal electrochemical sensors can include a unit array 100′ of solid microneedles on the substrate 180, where the plurality of microneedles 132, 134, 136, 138 include a solid body to form their respective microneedle structure. In such embodiments, the electrode structure can be a coating attached to at least the apex of the solid microneedle structure. The diagram of FIG. 1E illustrates this example of the electrode coating as coatings 141, 151, 161 and 171 for microneedles 132, 134, 136, and 138, respectively, which are configured over the apex of the respective microneedle. Yet, in some embodiments of the microneedle unit array 100′, the electrode coating (e.g., which can include the functionalization layers discussed for the first WE and/or second WE for HB and/or glucose sensing in accordance with the microneedle unit array 100) can be configured over an entirety or near-entirely of the microneedle's exterior wall. In various embodiments, an electrically-conductive material or portion of the electrode coating is electrically connected with the conduits or vias of the unit array 100′, e.g., which can be on top or within the substrate 180.

In some example embodiments of a reagentless, continuous epidermal electrochemical sensor device that includes the unit array 100 or 100′ of microneedles, the sensor device can be configured for detection of one or more diabetes biomarkers. For example, the sensor device includes a substrate; a plurality of microneedle electrodes coupled to the substrate and operable to penetrate within skin and contact the microneedle electrodes with interstitial fluid when the device is attached to skin of a user, each microneedle electrode including a microneedle structure and an electrode structure, wherein each microneedle structure includes an exterior wall spanning outward from a base surface of the microneedle structure and forming an apex at a terminus point of the exterior wall, and the electrode structure is configured within a hollow interior region of the microneedle structure or on at least a portion of the exterior wall of the microneedle structure; an enzymatic functionalization layer coupled to the electrode structure of the first microneedle electrode of the plurality of microneedle electrodes operable to detect β-hydroxybutyrate (HB) in the interstitial fluid through an electrochemically-mediated enzymatic reaction, wherein the enzymatic functionalization layer is immobilized to the electrode structure and comprises a β-hydroxybutyrate dehydrogenase (HBD) enzyme and HBD-cofactor that is unbound to the HBD enzyme; and a redox mediator coupled to the electrode structure of the first microneedle electrode to facilitate electron transfer in the electrochemically-mediated enzymatic reaction, wherein the plurality of microneedle electrodes includes a counter electrode or a reference electrode, or both, configured to apply or detect an electrical signal between the counter electrode and/or reference electrode and the first microneedle electrode. In some embodiments of the sensor device, the HBD-cofactor includes nicotinamide adenine dinucleotide (NAD+). For example, the senor device is engineered to have the enzymatic functionalization layer integrate the HBD enzyme and the NAD+ in a compact material such that the sensor device is operable to detect a level of HB in a reagentless electrochemical redox reaction, where HBD-catalyzed oxidation of the HB oxidizes to acetylacetate (AcAc) with a concomitant reduction of the NAD+ to reduce to nicotinamide adenine dinucleotide (NADH), and where the reduced NADH oxidizes back to the NAD+ based on the electron transfer facilitated by the redox mediator to continuously regenerate the HBD-cofactor. In some embodiments of the sensor device, the redox mediator comprises one or more of a phenazine, a phenothiazine, a phenoxazine, or a quinoid organic compounds to catalyze an oxidation reaction of a reduced species of the HBD-cofactor that oxidizes to the HBD-cofactor. For example, the redox mediator can include phenanthroline dione (PD), meldola blue, tetracyanoquinodimethane (TCNQ), or tetrathiofulvalene (TTF). In some embodiments, the sensor device can include an outer layer including one or more polymeric layers. For example, the polymeric layers) can include one or more of chitosan, polyvinylchloride (PVC), or a permselective or semipermeable polymeric membrane comprising at least one of Nafion, polyurethane, or cellulose acetate.

Further example features of the sensor device can include the following. For example, the redox mediator can be integrated in a material of the electrode structure of the first microneedle electrode. Examples of the electrode structure of the first microneedle electrode can include a carbon paste transducer comprising (i) one or more of graphite, carbon nanotubes, or graphene and (ii) a pasting liquid comprising one or more of ionic liquid (IL) or mineral oil.

In another example, the electrode structure of the first microneedle electrode can include a printable conductive ink, in which the enzymatic functionalization layer coupled to the electrode structure of the first microneedle electrode can also include a printable ink material entrapping the HBD enzyme and the HBD-cofactor within the printable ink material, such that the redox mediator is entrapped within one or both of the printable conductive ink and the printable ink material. For example, a multi-layer of ink coatings can be printed, e.g., layer-by-layer by an ink-jet printer, to form the various embodiments of the enzymatic functionalization layer, which comprises the electrode, the mediator, the enzyme co-factor, a stabilizer, a cross-linker, and/or a permselective protective coating.

In another example, the sensor device can integrate the enzymatic functionalization layer with the working electrode by redox hydrogel coating layer including the enzyme, the enzyme co-factor, and the mediator, each entrapped physically or wired chemically within a polymer layer. In some embodiments, for example, the enzymatic functionalization layer can include a hydrogel coating that entraps the HBD enzyme, the HBD-cofactor, and the redox mediator within a hydrogel material. In any example embodiments of the sensor device, the enzymatic functionalization layer is immobilized to the electrode structure of the first microneedle electrode by a cross-linking agent. For example, the cross-linking agent can include glutaraldehyde.

Further example features of the sensor device can include the following, particularly for multiple diabetes biomarker detection and continuous monitoring. For example, the sensor device can be configured to simultaneously monitor multiple diabetes biomarkers, such that the sensor device further includes a glucose-sensing enzymatic functionalization layer coupled to the electrode structure of a second microneedle electrode of the plurality of microneedle electrodes operable to detect glucose in the interstitial fluid through a glucose-sensing electrochemically-mediated enzymatic reaction, wherein the glucose-sensing enzymatic functionalization layer is immobilized to the electrode structure and comprises a glucose oxidase (GOx) enzyme and a mediator to facilitate electron transfer in a redox reaction. In some embodiments of the sensor device, for example, the mediator includes Prussian blue (PB). In some embodiments of the sensor device, for example, the glucose-sensing enzymatic functionalization layer further includes a permeable polymer film that immobilizes the GOx and the mediator to the electrode structure of the second microneedle electrode. In some embodiments of the sensor device, for example, the permeable polymer film comprises a one or more of chitosan, polyvinylchloride (PVC), or a permselective or semipermeable polymeric membrane comprising at least one of Nafion, polyurethane, or cellulose acetate.

Additionally, or alternatively, for example the sensor device can be configured to simultaneously monitor multiple diabetes biomarkers including lactate, where the sensor device further includes a lactate-sensing enzymatic functionalization layer coupled to the electrode structure of another (e.g., a second or a third) microneedle electrode of the plurality of microneedle electrodes operable to detect lactate in the interstitial fluid through a lactate-sensing electrochemically-mediated enzymatic reaction, wherein the lactate-sensing enzymatic functionalization layer is immobilized to the electrode structure and comprises a lactate oxidase (LOx) enzyme and a mediator to facilitate electron transfer in a redox reaction. In some embodiments of the sensor device, for example, the mediator includes Prussian blue (PB). In some embodiments of the sensor device, for example, the lactate-sensing enzymatic functionalization layer further includes a permeable polymer film that immobilizes the LOx and the mediator to the electrode structure of the third microneedle electrode. In some embodiments of the sensor device, for example, the permeable polymer film comprises a one or more of chitosan, polyvinylchloride (PVC), or a permselective or semipermeable polymeric membrane comprising at least one of Nafion, polyurethane, or cellulose acetate.

In any of the embodiments of the sensor device, for example, the counter electrode and/or the reference electrode includes carbon paste (CP) or an electrically conducive wire. In some embodiments of the sensor device, for example, the electrode structure is configured as a coating on at least a portion of the microneedles structure of the first microneedle electrode. In some embodiments of the sensor device, for example, the microneedle structure of the first microneedle electrode has an opening leading in to the hollow interior region of the microneedle structure that is defined by an interior wall, wherein the electrode structure is at least partially contained within the hollow region. In various embodiments of the sensor device, for example, the microneedle structure of each of the plurality of microneedle electrodes can include a pyramidal geometry, a conical geometry, or a combination thereof. Also, for example, the sensor device can include a plurality of electrical conduits, each coupled to the electrode structure of each of the microneedle electrodes and disposed on or within the substrate, wherein each electrical conduit terminates at an interface portion of the electrical conduit.

In some embodiments of the sensor device, for example, the sensor device includes an electrical circuit electrically connected to the plurality of electrical conduits to process the electrical signal as a processed signal. For example, the electrical circuit can include signal processing circuitry to amplify, filter and/or multiplex the detected electrical signal acquired by the microneedle electrodes of the sensor device; and/or the electrical circuit can include a data processing unit comprising a processor and memory to analyze the detected electrical signals and produce data associated with a detected analyte level, e.g., HB level, glucose level, lactate level, etc. In various embodiments, for example, the electrical circuit may be configured on the substrate or coupled to the substrate, such as in a single device packaging. Yet, in some embodiments, for example, the electrical circuit (or at least certain circuit modules) can be configured off-board of the substrate and receive signals for processing via a wireless transmitter. For example, in some embodiments, the sensor device includes a wireless transmitter in communication with the microelectrodes (e.g., directly or indirectly via basic modules of an electrical circuit) to transmit the detected electrical signals, e.g., raw signals and/or pre-processed signals.

In some implementations of the example embodiments of the devices of FIGS. 1A-1E, reagentless and continuous ketone bodies monitoring (CKM) using a microneedle platform with an array of microneedles can be demonstrated using the following chemicals: Ascorbic acid (AA), bovine serum albumin (BSA), calcium chloride anhydrous (CaCl₂), Chit (medium molecular weight), glacial acetic acid (HOAc), D-(+)-glucose anhydrous (GL), GOx (EC 1.1.3.4, 50 KU) from Aspergillus niger, GA (50 wt % solution), hydrochloric acid (HC1), HBD (EC 1.1.1.30, 25 UN) from Pseudomonas lemoignei, HB. Iron (III) chloride (FeCl3), magnesium sulfate anhydrous (MgSO4), mineral oil (MO), γ-globulins from bovine blood, NADH (nicotinamide adenine dinucleotide, reduced form), NAD+ (nicotinamide adenine dinucleotide, oxidized form), 1,10-phenanthroline-5,6-dione (PD), phosphate buffer solution (PBS) (1.0 M, pH 7.4), PVC, potassium chloride (KCl), potassium hexacyanoferrate (III) (K3Fe(CN)6), potassium phosphate monobasic (KH2PO4), potassium phosphate dibasic (K2HPO4), sodium bicarbonate (NaHCO3), sodium chloride (NaCl), sodium gluconate, sucrose, Tris-HCl buffer solution (1M), Triton X-100 and uric acid (UA). Ag wire (500 μm), acetone, tetrahydrofuran (THF), ethanol, Agarose and graphite powder (GP, crystalline 99%), 1-Ethyl-3-methylimidazolium bis(trifluoromethylsulfonyl)imide (IL). All reagents and solvents may be used without further modification and purification.

All electrochemical measurements (cyclic voltammetric (CV) and amperometric techniques) may be carried out in PBS (0.1 M, pH 7.4) during NADH analysis. Amperometric experiments may be performed in PBS (0.1 M, pH 7.4) and in artificial ISF (pH 7.4) at applied potentials of −0.1 V and 0.0 V for NADH and HB, respectively, and at −0.1 V for GL. The long-term stability measurements for NADH and HB detection may be carried out by embedding the microneedle patch inside a 3D printed 500 μM internal volume electrochemical cell. All the potentials are measured vs. Ag/AgCl wire-integrated microneedle electrode. The target analytes (HB and GL) may be freshly prepared in PBS or artificial ISF for all experiments.

In some implementations of the disclosed technology, the microneedle sensor arrays containing a plurality of microneedles, for example, four hollow microneedles with inter-needle spacing of 1 mm and internal diameter of 500 μm may be fabricated based on some embodiments of the disclosed computer numerical control (CNC)-micromachining technique. In some implementations of the disclosed technology, two of these hollow microneedles may be used, after carbon paste (CP) packing and subsequent modification, as working electrodes (WEs) for HB and GL sensing platforms in case of simultaneous dual-analyte HB/GL detection, which otherwise the electrode for non-measuring analyte left unmodified (i.e. in case of single-analyte detection). In some implementations of the disclosed technology, counter electrode (CE) may be a CP-filled hollow microneedle. In some implementations of the disclosed technology, reference electrode (RE) may be constructed by embedding a hollow microneedle with a 5 mm-long Ag wire, followed by sealing from rear side with a photocurable resin, UV irradiation for 2 min to cure the resin, and chloridation of the Ag wire by dropping 10 μL FeCl₃ (0.1 M) for 1 min and washing with water. To establish the electrical contacts from the rear side of the microneedles, the stainless copper wires may be individually connected by using conductive silver epoxy ink and cured at 75° C. for 15 min before CP-packing and further surface modification on each microneedle (WE1, WE2). The CP used to form the HB sensor may be prepared by mixing 65 wt % graphite powder (GP) and 35 wt % ionic liquid (IL) followed by the addition of 50 uL PD solution (10 mg mL⁻¹) dissolved in 1:1 ethanol/acetone (4 mg GP/IL:1 μL PD). HBD stock solution may be prepared by dissolved the lyophilized powder in 0.1 M Tris-HCl buffer solution (pH 8.5) containing 0.1 wt % BSA and aliquoted and frozen for further use. NAD⁺ powder may also be aliquoted to avoid frequent thawing/freezing cycles. A 0.5 wt % Chit solution may be prepared by dissolving 25 mg Chit powder in 5 mL of 1 wt % HOAc solution. The PVC-nonionic surfactant solution may be obtained by dissolving 120 mg PVC in THF solvent followed by adding 5 μL of Triton X-100. The mixed HBD/NAD⁺ solution may be prepared by mixing NAD⁺ solution (0.5 M, dissolved in 0.1 M Tris-HCl buffer (pH 8.5, 0.1 wt % BSA)) with 1 mg mL⁻¹ HBD solution (1:2). After CP-packing and smoothing its surface with a surgical blade, the WE1 may be drop casted by 1 μL of HBD/NAD⁺mixed solution, followed by 0.5 μL of 2% GA solution and 0.5 μL of 0.5% Chit solution, respectively. Finally, 0.5 μL PVC solution may be cast onto the microneedle surface and kept overnight in the refrigerator for further HB sensing experiments.

In some implementations, the microneedle WE2 as GL microneedle sensing platform may be fabricated by packing the hollow microneedle with the CP based on a 65 wt % GP and 35 wt % MO composition. After smoothing the surface with a surgical blade, Prussian Blue (PB) may be electrodeposited by applying CV technique in an acidic 0.1 M HCl solution containing 0.1 M KCl, 2.5 mM K3Fe(CN)6 and 2.5 mM FeCl3 over the potential range of −0.5 to 0.6 V for 10 cycles at scan rate of 50 mV s−1. To stabilize the deposited PB film, the microneedle sensor may be cured at 100° C. for an hour. Then, the microneedle WE2 surface may be dropped by 1 of 5 U μL−1 GOx solution prepared in PBS (0.1 M, pH 7.4 with 10 mg mL−1 BSA), followed by 0.5 μL of 2% GA solution, 0.5 μL of 0.5% Chit solution, 0.5 μL of the PVC solution and ultimately kept overnight in the refrigerator for further future experimentation of GL sensing.

In some implementations, the artificial ISF used as the analysis medium may be prepared based on some embodiments of the disclosed technology. For NADH analysis, CV experiments may be carried out in the potential range of −0.5 to 0.3 V and amperometric experiments may be examined for 90 s at −0.1 V in 0.1 M PBS (pH 7.4). To demonstrate real-time CKM and dual-analyte HB/GL simultaneous detection, the amperometric experiments may be operated at applied potential of 0.0 V for HB detection within 90 s and −0.1 V for GL detection within 60 s in artificial ISF. The selectivity of the developed CKM microneedle device may be tested in artificial ISF in the presence of possible interfering species such as ascorbic acid (AA), uric acid (UA) and acetaminophen (AP). The long-term stability and anti-biofouling characteristics of the CKM system may be investigated in artificial ISF containing proteins (BSA and globulins at 4 mg mL−1 each) by repetitive amperometric operation every 10 min for a prolonged period more than 6 hours (380 min). Additionally, the cross-talk investigation of dual-analyte HB/GL simultaneous detection may be operated on the developed microneedle sensor array for each individual sensing platform in artificial ISF.

In some implementations, a 1.4% agarose gel covered by a layer of parafilm may be used as phantom gel skin-mimicking model. Firstly, 140 mg of agarose powder may be poured into 10 mL of artificial ISF and heated at 200° C. under continuous stirring condition until the liquid turned homogenous and completely clear. The solution may be poured into circled-shaped molds and allowed to solidify within few minutes at room temperature. Each different ketone concentrations (0, 2, 4 and 6 mM HB) may be individually injected into each obtained phantom gel and allowed to equilibrate overnight while kept in refrigerator. The capability of the CKM microneedle device to detect HB in skin-mimic model may be performed by pressing the microneedle through the parafilm-covered gel and running amperometry at 0 V within 90 seconds.

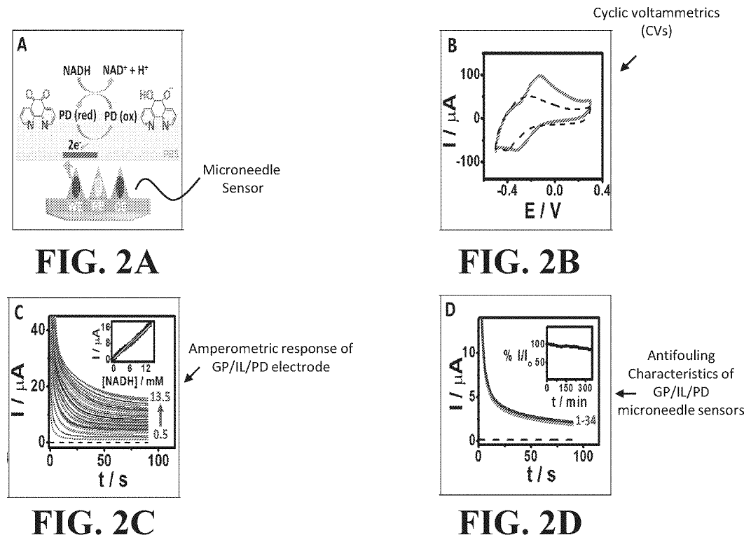

FIGS. 2A-2D show NADH (nicotinamide adenine dinucleotide, reduced form) electrocatalysis analysis and example data using an example ionic liquid (IL)-based carbon paste (CP) microneedle sensor platform. Specifically, FIG. 2A shows the schematic diagram of phenanthroline dione (PD)-mediated NADH electrocatalysis on the microneedle sensor, FIG. 2B shows cyclic voltammetrics (CVs) showing the low-potential NADH catalysis on the IL-prepared graphite carbon paste microneedle sensor containing 0.25% PD. CVs may be recorded in PBS (0.1 M, pH 7.4) before and after addition of 8 mM NADH, FIG. 2C shows amperometric response of graphite powder (GP)/ionic liquid (IL)/phenanthroline dione (PD) electrode to successive 0.5 mM additions of NADH at potential −0.1V vs. Ag/AgCl with the corresponding calibration plot for NADH analysis shown as the inset, and FIG. 2D shows antifouling characteristics of GP/IL/PD microneedle sensors toward continuous measurement of 2 mM NADH addition in 10 min time intervals. Amperometric responses relate to 34 measurements of NADH recorded in a 3D printed cell. Inset shows the relative current change during more than 5 h.

In some implementations, PD can be used as the mediator molecule can offer unique advantages, such as high insolubility in aqueous solution, which addresses the issue of mediator leaching and sensor instability, and good compatibility with β-hydroxybutyrate dehydrogenase (HBD) enzyme. Also IL-based CP packed microneedle electrode provides antifouling properties and also more sensitivity toward NADH detection over extended periods of time (>5 hr). The invention leads to a stable confinement of the NAD+ cofactor and to a stable detection of the NADH product.

In some implementations, the NAD-dependent dehydrogenases constitute the largest group of redox enzymes known today and relate to a vast number of substrates. As they do not have the oxygen dependency issue, which is a common problem in oxidase-based enzymes, the development of a real-time assay based on this family of enzymes will open up a key avenue to develop more efficient clinical diagnosis systems. Toward this end, the first crucial step in designing dehydrogenase-based biosensors is to choose a suitable electrocatalyst mediator, capable of lowering the potential of NADH electrooxidation to the desired range of −0.2V to 0.0 vs. Ag/AgCl, where contributions to the response from competing oxidizable species such as AA, UA and AP are negligible, where electrochemical reduction of molecular oxygen is avoided, and where the potential of zero charge is found for most electrode materials, resulting in low background current and noise. The mediator molecule should also possess a high electron transfer rate and a preferentially selective reaction with NADH to yield the enzymatically active NAD+ as the end product. Another highly important factor is the compatibility of the mediator compound with the specific dehydrogenase enzyme. The common classes of quinoid NADH redox mediators in terms of their compatibility with the HBD enzyme toward developing a commercial amperometric HB blood biosensor show that while some common NADH mediators like Meldola Blue inhibit the HBD enzyme activity through covalent binding to important thiol groups in the enzyme structure, the phenanthroline quinone derivatives such as PD can overcome this mode of inhibition. This is due to the presence of reactive quinone double bonds into their heteroaromatic rings which avoid 1,4-nucleophilic addition with enzyme amino acid residues such as Cys. Based on these and studies using several mediator compounds, PD may be chosen as the mediator in our work not only due to its compatibility with the HBD enzyme but also because of its high insolubility which is essential to realize a continuous monitoring system with minimum leaching and thereby, high long-term stability.

The schematic of the NADH electrooxidation mechanism on the microneedle electrode based on the 2-electron-1-proton accepting PD mediator system is shown in FIG. 2A. The recorded cyclic voltammetrics (CVs) on the IL-prepared CP-filled microneedle electrode in PBS (phosphate buffer solution) buffer indicated that the NADH electrocatalysis starts at −0.26 V and reaches its peak current at −0.13V (FIG. 2B), showing the high capability of the proposed system for NADH oxidation. FIG. 2C presents the amperometric response of the IL-prepared CP-filled microneedle electrode to the successive additions of 0.5 mM NADH under low potential of −0.1 V, showing the anodic current response linearly increases upon NADH addition up to 13.5 mM. The NADH oxidation may also be performed on the MO-prepared CP microneedle sensors. As illustrated from both CVs and amperometric curves, much lower analytical performance is seen for nonconductive MO compared to the IL system. NADH detection on IL-prepared CP microneedle yielded an almost 4-fold higher sensitivity in comparison to that of MO-based CP electrode. Such higher sensitivity could be attributed to the fact that conductive IL paste can act as a better carrier of electrons and thus has higher charge transfer rate when compared to nonconductive MO-based electrode transducer. In addition, while the current response on the MO system saturates at 3.5 mM of NADH, the linear detection range on the IL system extends up to 13.5 mM, which exhibits resistance of IL against surface fouling. The excellent antifouling characteristics of the IL-based CP microneedle system may be further assessed by repetitive chronoamperometric measurements of NADH in 10 min time intervals during more than 5 h performed by embedding the microneedle in a 3D printed 500 μL electrochemical cell (FIG. 2D). The obtained results highlighted the promising antifouling nature of IL system with retaining 92% and 85% of its initial signal response after 240 min and 340 min, respectively. The superior performance of IL toward constructing stable and sensitive electrochemical transducers may also be demonstrated.

A critical requirement toward realizing the NAD-dependent dehydrogenase-based continuous monitoring devices is to efficiently constrain the NAD+ molecule in close proximity to the enzyme, to the mediator and to the electrode surface to ensure efficient electron transfer among the biosensor components. The first attempts to confine NAD+ onto the electrode surface were performed in late 1970s, including the use of acetylated dialysis membrane or binding to agarose and fixing the physical membranes on the surface of electrodes. These pioneering early examples however cannot be adapted to the miniaturized electrodes for implantation or in-vivo measurement purposes. These works inspired other groups to devise approaches for incorporating NAD+ to the electrode matrix, including mixing all the components within carbon paste electrode, covalent attachment of N1-carboxymethyl-NAD+ species to polyamino-saccharide chains of Chit and cross-linking with GA, noncovalent attachment of NAD+ to carbon nanotubes based on the π-π stacking interaction, and self-assembly process of NAD+ and Tb3+ to form 3D conducting polymeric nanoparticles. These reports while show interesting promises, however they failed to successfully address the issue of NAD+ confinement toward realization of a stable continuous monitoring system. The simple mixing of the enzyme and mediator into the CP matrix needs large amounts of enzyme reagent and also cannot prevent the reagents from leaching. The covalent tethering of enzyme/cofactor requires complex, time-consuming procedures including separation of unreacted chemicals by dialysis membrane and also does not provide enough mobility for the components to freely diffuse and undergo the electron transfer reactions. Furthermore, it limits the loading amount of the components. The noncovalent attachment through hydrophobic interactions similarly does not offer enough amount of the reagents for an efficient cofactor regeneration and also involves using of nanomaterials such as carbon nanotubes which their biocompatibility is a concern for in-vivo measurement. Ultimately, encapsulating the components within the structure of polymeric nanoparticles as the only system that exhibited a stable response for 5000 s of analysis under flow conditions, has not been tried toward long-term continuous monitoring.

Toward realizing a reagentless biosensor capable of real-time continuous monitoring of HB, we developed an efficient multilayer modification protocol benefitting from a synergistic integration of carefully selected components. Drop casting an optimized concentration ratio of enzyme/cofactor onto the PD-incorporated IL-based CP microneedle electrode allowed the biomolecules to diffuse inside the available surface pores of the underlying electrode, leading to their physical adsorption as well as hydrogen bonding and electrostatic interactions with the incorporated IL. This step may be followed by crosslinking the reactive amine groups in the enzyme/cofactor molecular structures, which creates an extended 3D polymer network including HBD/NAD+ biomolecules connecting through the pores of the electrode. Further confinement of the biomolecules may be achieved by utilizing outer polymer membranes. Various permselective and semipermeable natural or synthetic polymers have been introduced into the biosensors design, with the polyurethane, cellulose acetate, Nafion, PEG and Chit being the most common membranes in the literature. While testing different combination of membranes in our work, successive layers of Chit and PVC revealed the best performance toward both sensitive and stable continuous HB analysis. The high resistance of the PVC layer against biofouling in biosensor applications shows that modification of PVC with non-ionic surfactants reduce the biofouling and broadens the linear range for a whole blood lactate enzymatic sensor. The PVC/Chit-modified HB microneedle biosensor may be evaluated first in PBS medium. The effect of polymer layers on the analytical performance of the HB biosensor can be shown while the amperometric measurements are carried out at low potential of 0.0V. While Chit layer provided a linear calibration range up to 8 mM of HB concentrations, its long-term stability showed 35% decrease after 4 hours of continuous measurement in 4 mM HB solution. On the other hand, PVC-modified biosensor exhibited a stable current response after 4 hours, but the biosensor nearly saturated at 6 mM HB concentrations. However, by combining Chit and PVC, the HB biosensor revealed a linear calibration range in the entire range of tested HB concentrations up to 12 mM and also a stable current response during 4 h of HB analysis. In addition, the lower potential of amperometric HB detection compared to 0.2 V for HB biosensor using the PD mediator shows the superior performance of the underlying transducer implemented based on some embodiments of the disclosed technology.

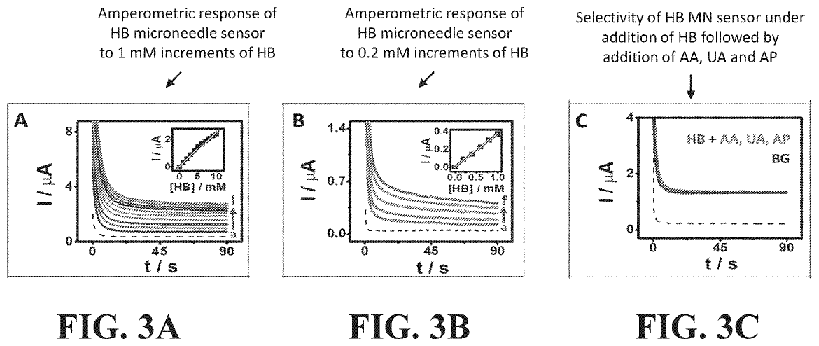

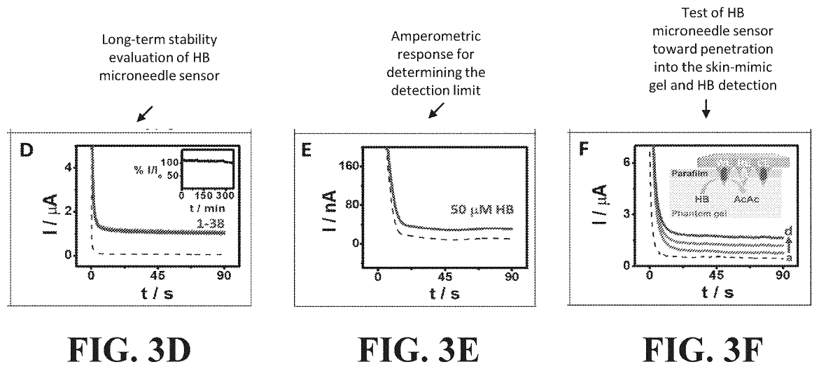

FIGS. 3A-3F show example data from a performance evaluation of an example embodiment of the HB microneedle sensor. Specifically, FIG. 3A shows amperometric response of HB microneedle sensor to 1 mM increments of HB in artificial ISF, FIG. 3B shows amperometric response of HB MN sensor to 0.2 mM increments of HB in artificial ISF, FIG. 3C shows the selectivity test of HB MN sensor under addition of 4 mM HB followed by 200 μM additions of AA (ascorbic acid), UA (uric acid), and AP (acetaminophen), FIG. 3D shows the long-term stability evaluation of HB microneedle sensor recorded in a 3D printed electrochemical cell every 10 min toward continuous monitoring of 4 mM HB in artificial ISF matrix, FIG. 3E shows the amperometric response for determining the detection limit, and FIG. 3F shows test of HB microneedle sensor toward penetration into the skin-mimic gel and HB detection in the range of 0-6 mM (a-d) and in 2 mM increments. Inset shows schematic representation of the experimental setup used to assess the electrochemical performance of HB microneedle in phantom gel medium. Parafilm may be used to simulate the outer layer of the skin, stratum corneum, and 1.4% agarose gel used as dermis and epidermis simulant. All the experiments may be performed under applied potential of 0.0 V vs. Ag/AgCl.

The successful confinement of the biosensor components is achieved through a layer-by-layer modification protocol, where HBD enzyme and NAD+ cofactor immobilized on the surface of microneedle sensors by using glutaraldehyde (GA) crosslinking agent, followed by drop casting chitosan and PVC/non-ionic surfactant polymer layers. Such an engineered layer-by-layer architecture ensures forming a 3D network which can prevent leaching of the enzyme/cofactor and at the same time providing a favorable environment for the electron transfer to occur. In addition, the permselective and anti-biofouling characteristics of the employed polymeric layers minimizes the foreign body response and nonspecific protein adsorption when applied for in-vivo continuous chemical analysis. Furthermore, the sensitivity and linear range of the biosensor is highly adjustable through manipulating the thickness of the polymeric permselective layers. The microneedle continuous ketone bodies monitoring (CKM) device may be investigated toward the HB analysis in skin-mimicking phantom gel and it has been shown that microneedle can easily penetrate the skin and analyze the HB concentration.

A sensor to be used reliably for interstitial fluid (ISF) continuous monitoring, needs to excel in all areas of analytical detection: dynamic range, sensitivity, selectivity, stability, response time, and biocompatibility. For low molecular weight species like glucose, the ISF concentration is very similar to the plasma concentration. There is no data on the ISF levels of HB, but its smaller size compared to the glucose suggests a near unity plasma/ISF ratio. This similarity in concentrations is due to the fast paracellular diffusion through the capillary walls compared to the turnover rate for ISF from the dermis. Regarding the plasma HB concentration, HB levels of <0.6 mM are considered normal, and when HB levels are in the range of 0.6-1.5 mM, both GL and HB levels are rechecked in 2-4 hours. Increasing HB levels to 1.5-3.0 mM is considered to be a sign of developing DKA while higher than 3.0 mM HB needs immediate emergency treatment. Therefore, a wide linear HB detection range covering both μM and mM concentrations is essential for realizing a real-time CKM in ISF. To this end, the analytical performance of the optimized multilayer-modified HB microneedle sensor may be evaluated in artificial ISF. The dynamic concentration range may be first examined in response to increasing levels of HB over the 1.0-10.0 mM HB range. FIG. 3A presents chronoamperograms for increasing concentrations of HB in 1.0 mM increments (b-n) in artificial ISF medium. As illustrated, the HB microneedle biosensor displayed well-defined chronoamperograms with current signals being proportional to the HB concentration. The resulting calibration plot (shown in the inset) exhibited high linearity (slope: 0.263 μA mM−1; correlation coefficient: 0.990). In addition, the remarkable low background current (a) which is associated with the low operating potential. Similarly, the detection of μM concentration range of HB may be also investigated by the HB microneedle biosensor, with the obtained amperometric curves shown in FIG. 3B. The amperograms may be obtained in the range of 0.0-1.0 mM with successive additions of 0.2 mM HB, showing linearly increasing current response upon HB addition. These data indicate that the developed HB microneedle sensor features a high sensitivity toward HB detection in the whole desired concentration range of the analyte.

Since the microneedle biosensor is expected to be exposed to complex matrix of ISF, it should offer selective response in the presence of other competing electroactive constituents of the ISF. The selectivity may be evaluated in the presence of physiological levels of the relevant electroactive constituents of human ISF, 200 μM for AA, UA and AP. FIG. 3C shows the chronoamperometric response for 4.0 mM of HB in the presence and absence of such physiological concentrations of AA, UA and AP. These data clearly indicate that these potential interferences have a negligible effect upon the HB response and hence the new HB microneedle biosensor system offers high selectivity. Such a high selectivity of the biosensor can be mainly attributed to the very low detection potential offered by the PD mediating system.

The dehydrogenase-based continuous monitoring system implemented based on some embodiments of the disclosed technology, as discussed above in detail, may achieve a high operational stability for a prolonged period of analysis. The stability of the microneedle sensor may be tested in 4 mM HB solution for 380 min at each 10 min intervals in artificial ISF. FIG. 3D shows the corresponding chronoamperograms along with the time-course profile of the resulting current response (inset: with the initial result at t=0 min normalized to 100%). The obtained data indicates a highly stable current response over the entire time of operation (>6 h), with retaining 95% of the initial current response. Such long-term stability in artificial ISF medium in the presence of highly passivating proteins may be achieved through synergistic combination of several factors in the design and operation of the developed microneedle sensor; (i) the established modification protocol efficiently and stably confines the various components of the sensing system, without affecting their functions to efficiently undergo the charge transfer reactions; (ii) a very low operating voltage that selectively oxidizes HB, but does not promote the formation of passivating polymeric films; (iii) a smart combination of Chit and PVC-surfactant as outer polymeric layers with semipermeability and permselectivity properties, respectively, which can provide a sieving characteristics to the sensor based on charge and size of the components in the medium and thereby, offers excellent anti-biofouling features to the system.

FIG. 3E shows that the detection limit of the developed HB microneedle sensor in artificial ISF medium is 50 μM (S/N=3), which shows the high capability of the microneedle-based sensor toward detecting even very low levels of HB. The herein achieved limit of detection is quite lower in comparison to other previously reported HB sensors.

To predict the performance of the microneedle biosensor under operating in real scenario case of on-body testing, a skin-mimicking phantom gel may be prepared based on the protocol established by Chang et al, by covering a 1.4% agarose hydrogel (mimicking dermis with ISF) with a parafilm layer (representing the water-impermeable stratum corneum and epidermis) (FIG. 3F; inset). The tips of the microneedle sensor array are positioned inside the phantom gel by pressing the patch through the parafilm layer, after which chronoamperometry may be performed to capture the signal response. As shown in FIG. 3F, the tests in various skin-mimicking phantom gels containing various HB concentrations of 0, 2, 4, and 6 mM resulted in well-defined amperometric responses, with increasing current upon increasing HB levels. This reflects the suitability of such a microneedle sensing platform toward transdermal and skin-through sensing operations.

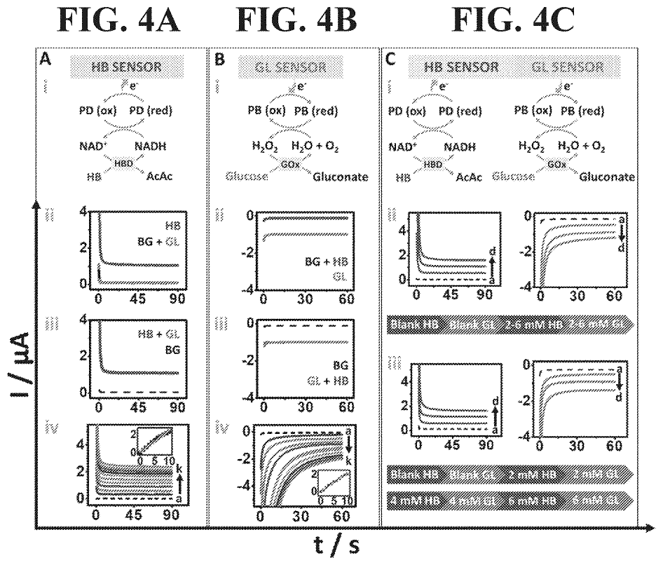

FIGS. 4A-4C show cross-talk study towards simultaneous HB/GL dual-analyte measurements. Specifically, FIG. 4A shows investigation of cross-talk on HB microneedle sensor, FIG. 4B shows investigation of cross-talk on GL microneedle sensor, and FIG. 4C shows simultaneous dual-analyte HB/GL detection on the adjacent electrodes of a single microneedle sensor performed in a single drop of artificial ISF.

FIG. 4A shows investigation of cross-talk on HB microneedle sensor as follows: (i) Schematic representation of the HB analysis on the microneedle electrode; (ii) Amperometric curves of blank measurement in artificial ISF (BG), followed by 4 successive additions of 1 mM GL and a 4 mM HB addition; (iii) Amperometric response recorded in artificial ISF (BG) and after addition of 4 mM HB, followed by four successive 1 mM GL additions; (iv) Amperometric response of the HB microneedle sensor to successive additions of HB/GL mixture in 1 mM increments over HB/GL concentration range 1-10 mM (b-k). The corresponding calibration plot has been shown as inset.

FIG. 4B shows investigation of cross-talk on GL microneedle sensor as follows: (i) Schematic showing the GL analysis on the microneedle sensor; (ii) Amperometric curves of blank measurement in artificial ISF (BG), followed by 4 successive additions of 1 mM HB and a 4 mM GL addition; (iii) Amperometric response of GL microneedle sensor in artificial ISF (BG) and after 4 mM GL followed by four successive 1 mM additions of HB (x-y); (iv) Chronoamperometry obtained on GL MN sensor upon successive additions of glucose/HB mixture in 1 mM increments over HB/GL concentration range 1-10 mM (b-k).

FIG. 4C shows simultaneous dual-analyte HB/GL detection on the adjacent electrodes of a single microneedle sensor performed in a single drop of artificial ISF as follows: (i) Schematic representation of the simultaneous dual-analyte detection; (ii) Amperometric response of the HB/GL microneedle sensor to successive additions of 2-6 mM HB and GL (b-d), respectively. The order of recording the amperograms has been shown below the curves; (iii) Amperometric responses of the HB/GL microneedle sensor to 2 mM HB, 2 mM GL, 4 mM HB, 4 mM GL, 6 mM HB and 6 mM GL (b-d). GL amperometric measurements may be carried out under Eappl.=−0.1 V while the HB amperometric detection may be performed at 0.0 V vs. Ag/AgCl.

The simultaneous detection of ketone bodies along with glucose on a same microneedle platform would offer a major advance toward improving the life of the people afflicted with diabetes. The integration of two fundamentally different enzymatic biocatalytic systems based on dehydrogenase-oxidase chemistry on a single sensing platform is a quite challenging task and needs a carefully thought fabrication and detection protocol for each analyte. The presence of each analyte may interfere during the selective detection of the other analyte and may be a major concern toward the fabrication of multiplexed sensor with minimal false alarms. Therefore, the microneedle sensors of HB and GL is first prepared, and the cross-talk during the detection procedures is individually investigated for each sensing platform in artificial ISF medium, as shown in FIGS. 4A-4C. The detection mechanism and the cross-talk investigation for HB microneedle biosensor is shown in FIG. 4A (i)-(iv). FIG. 4A(ii) shows that four successive additions of 1 mM glucose does not change the current signal of HB biosensor but an addition of 4 mM HB gives a jump in current signal, indicating that HB can be selectively detected in the presence of GL. The experiment may also be performed by first spiking a 4 mM HB to artificial ISF followed by four successive 1 mM spikes of GL (FIG. 4A(iii)). As illustrated in FIGS. 4A-4C, GL additions do not affect the current signal which shows that GL cannot react with HB oxidation products and thus, cannot influence the sensing performance of HB microneedle. The successive additions of HB/GL mixture into the artificial ISF in 1 mM increments of each analyte may be performed and the results is shown in FIG. 4A(iv). The obtained calibration plot in the presence of GL (shown as inset) revealed a linear dynamic range for HB detection over the investigated range of 1-10 mM with a quite similar sensitivity to the calibration plot for HB alone in FIG. 3A (0.25 μA mM−1 vs. 0.26 μA mM−1).

In some implementations, the GL microneedle biosensor may be constructed through a multilayer deposition protocol in which GOx may be immobilized by GA crosslinking onto the PB deposited CP-packed hollow microneedles, followed by coating with Chit and PVC as outer membranes (FIG. 4B (i)). The performance of as-prepared microneedle WE, assessed in PBS (0.1 M, pH 7.4) at applied potential of −0.1 V vs. Ag/AgCl integrated-wire microneedle RE, illustrated its high capability toward sensitive GL detection over the concentration range of 0.5-12.0 mM. Similar to FIG. 4A, the cross-reactivity of analytes during GL detection on the microneedle sensor may be tested (FIG. 4B (ii-iv)). FIG. 4B(ii) shows the response of the GL microneedle sensors to four successive additions of 1 mM HB followed by an addition of 4 mM GL. The GL sensor is not responding to the HB addition while resulting in a well-defined amperometric signal to 4 mM GL addition, reflecting the reduction of (H₂O₂) enzymatic product of GL oxidation by PB transducer at −0.1 V vs. Ag/AgCl. This indicated that GL can be selectively detected in the presence of HB. Similarly, chronoamperometric curves obtained by spiking a 4 mM GL to artificial ISF followed by four successive 1 mM spikes of HB well demonstrated that HB analyte does not undergo any reaction with the products resulted from the bioelectrocatalytic determination of GL (FIG. 4B(iii)). The successive additions of 1 mM increments of HB/GL mixture onto the GL microneedle sensor may also be carried out in the artificial ISF, and the resulting calibration plot in FIG. 4B(iv, inset) demonstrated a linear dynamic range over 1-10 mM of GL (0.036 μA mM−1; correlation coefficient 0.995) with clearly no cross-reactivity from HB.

The electrochemical microneedle platform implemented based on some embodiments of the disclosed technology can be used to provide the simultaneous dual-marker HB/GL continuous monitoring in the ISF body fluid over long periods of time. The HB and GL biosensors may be fabricated on adjacent microneedles and the detection may be performed in a single droplet of artificial ISF sample (FIG. 4C(i)). The potential of the HB/GL microneedle array for such simultaneous detection is demonstrated in FIG. 4C(ii, iii). The order of spiking the analytes for each experiment is also shown. The resulting chronoamperograms demonstrated an attractive performance of the multiplexed microneedle-based sensor toward simultaneous detection of HB and GL to yield a well-defined response with no cross talk between the two analytes at the neighboring microneedles. Overall, the data of FIG. 4 clearly demonstrates that the design of the individual HB and GL microneedle offers efficient detection of these diabetes markers, without cross talk or potential leaching which can potentially interfere with the detection of other analyte on the neighboring electrode.

In some implementations of the disclosed technology, a single microneedle device can provide the simultaneous multiplexed HB/GL detection while the HB working electrode is based on the HBD/NAD+ enzyme/cofactor mixture and the oxidation of the resultant NADH enzymatic product on the graphite/IL/PD microneedle and the GL detection relies on the glucose oxidase (GOx) enzyme immobilized working electrode and the reduction of the produced hydrogen peroxide ((H₂O₂)) on the Prussian blue (PB) modified graphite/mineral oil CP electrode. The obtained results show no cross-talk between the HB and GL dual analytes.

In some implementations of the disclosed technology, the CKM microneedle sensor platform and multiplexed HB/GL microneedle sensing device can be used to detect HB within ISF samples. In some implementations of the disclosed technology, the CKM microneedle sensor platform and multiplexed HB/GL microneedle sensing device may be used to detect HB/GL simultaneously. Also, in some implementations, the device may be implemented as a fully-integrated microneedle patch to be worn on body. In addition, the disclosed technology can be implemented in some embodiments to detect other diabetic biomarkers using a single sensing device. The novel protocol for the continuous monitoring of analytes based on NAD-dependent dehydrogenase enzyme has opened up new possibilities for real-time assessment of other important analytes using the corresponding dehydrogenase enzyme. Hence, a number of variations may be made in the disclosed embodiments without departing from the scope of the disclosed technology to provide new sensors, along with glucose, for improving the glycemic control of diabetes patients.

Considering the tremendous commercial promise of diabetes management, the commercial scope of the new device is enormous. The CKM device implemented based on some embodiments can be used in the monitoring of DK/DKA, especially for type I diabetic patients who are in the need of insulin injections. Such use can be in forms of fully-integrated self-monitoring wearable microneedle patches and/or use by health providers. Nowadays, there is a growing interest on the importance of the other biomarkers detection along with GL. HB which is the dominant form of ketone bodies is highly essential to monitor in parallel with glucose. The capability to measure rising levels of both glucose and ketone bodies will provide the diabetics with direct information on the need to take additional insulin or seek medical advice. The synthesis of ketone bodies is finely regulated by the balance between the anabolic hormone insulin and the catabolic hormones adrenaline, noradrenaline, cortisol and glucagon. This balance may be altered in certain endocrine disorders such as insulin-dependent diabetes mellitus, in which ketone body levels are often mildly elevated (DK). At times of stress or infection or upon poor compliance to the insulin regime, severe hyperketonaemia supervenes with resultant acidaemia (DKA). It has been shown that before raising the glucose concentration, the ketones level increases apparently after the interruption of insulin administration; thus measuring ketone bodies can represent a useful mean for early diagnosis of insulin deprivation. Currently, monitoring of DK/DKA relies solely in-vitro on blood test strips available for GL and for HB detection that are implemented by extracting blood through finger pricking and the use of disposable strips. The disclosed technology can be implemented in some embodiments to provide an on-body sensor for continuous realtime CKM monitoring. Such continuous CKM offers great promise also for laboratory-based (e.g., hospital) clinical flow-based analyzers.

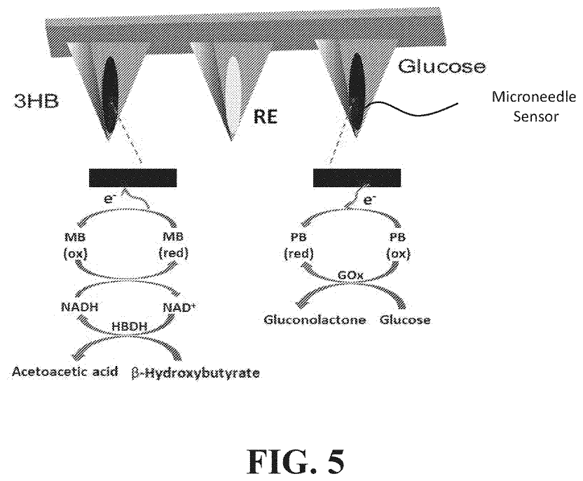

FIG. 5 shows an example schematic of microneedle-based electrochemical biosensor for real-time monitoring of dual glucose/beta-hydroxybutyrate (BHB) markers in ISF. In some implementations, the microneedle-based electrochemical biosensor may include an array of microneedle sensors including a plurality of protruded needle structures each including a hollow interior and a plurality of electrodes arranged in the hollow interiors of the plurality of protruded needle structure, respectively. The plurality of electrodes may include a first working electrode structured to interact with beta-hydroxybutyrate such as 3-β-hydroxybutyrate (3HB) and a second working electrode structured to interact with glucose. The plurality of electrodes may also include a reference electrode to provide a reference electrical potential to the plurality of protruded needle structures including the first and second working electrodes. The first and second working electrodes are configured to detect the first and second chemical or biological substance simultaneously, and an electrical potential difference between the reference electrical potential and the first and second working electrodes are measured.

The disclosed technology can be implemented in some embodiments to create a wearable minimally-invasive system for continuous monitoring of diabetic ketoacidosis (DKA). DKA is a serious complication of diabetes that requires prompt medical attention. The proposed approach is based on the simultaneous minimally-invasive monitoring of glucose and beta-hydroxybutyrate (BHB, as dominant form of ketone bodies) in ISF.

The microneedle monitoring of BHB relies on a NAD+-dependent dehydrogenase enzyme (HBDH) which is more challenging for reagentless continuous microneedle operation. A) Retention of the NAD+ cofactor; B) Stable NADH detection without surface fouling.

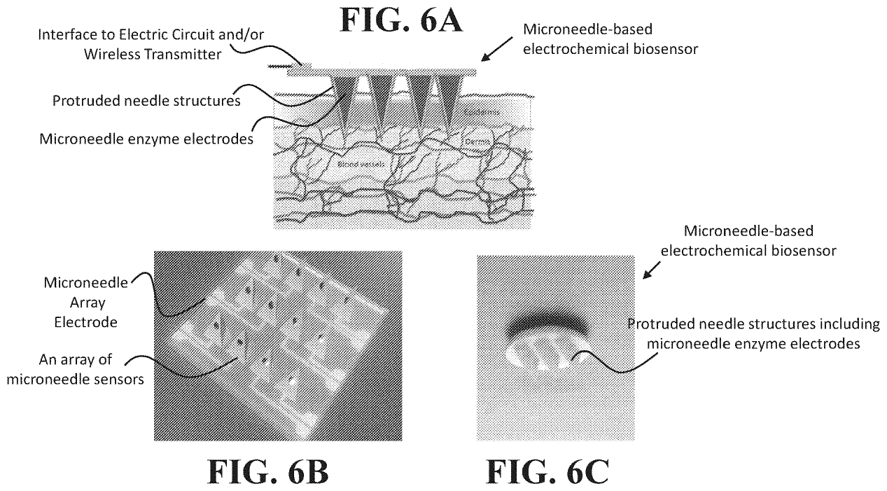

FIGS. 6A-6C show examples of microneedle-based electrochemical biosensor based on some implementations of the disclosed technology. The disclosed technology can be implemented in some embodiments to provide microneedles for transdermal monitoring of ISF biomarkers, enabling the multi-analyte chemical sensing. In some implementations, the microneedle-based electrochemical biosensor may include an array of microneedle sensors including a plurality of protruded needle structures each including a hollow interior and a plurality of microneedle enzyme electrodes arranged in the hollow interiors of the plurality of protruded needle structure, respectively. The microneedle enzyme electrodes implemented based on some embodiments of the disclosed technology can be used to achieve continuous real-time minimally invasive monitoring of multiple chemical markers.

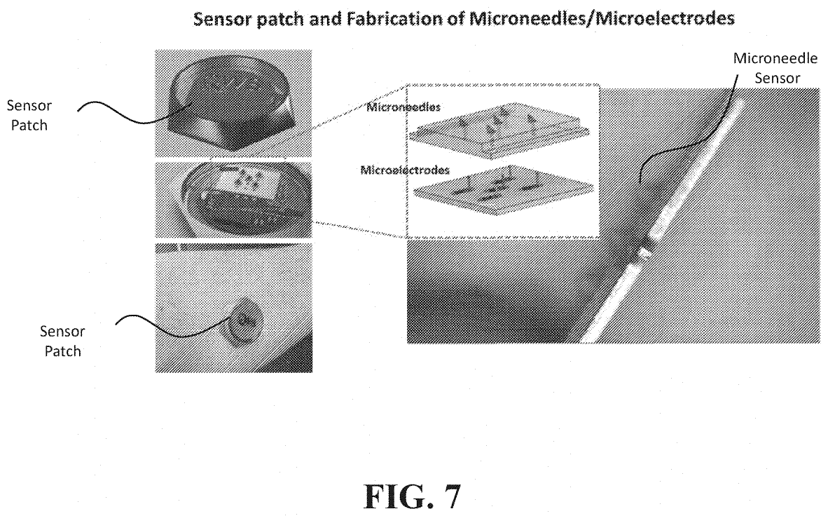

FIG. 7 shows an example of sensor patch that shows electronic interface and integration implemented based on some embodiments of the disclosed technology. In some implementations, a dual-potentiostatic electronic chip may be mounted over the microneedle patch for providing control of the microneedle sensors along with wireless transmission of the corresponding signals. In one example, the microneedle sensors may include hollow microneedles and aligned pillar-electrodes inserted into the hollow microneedles.

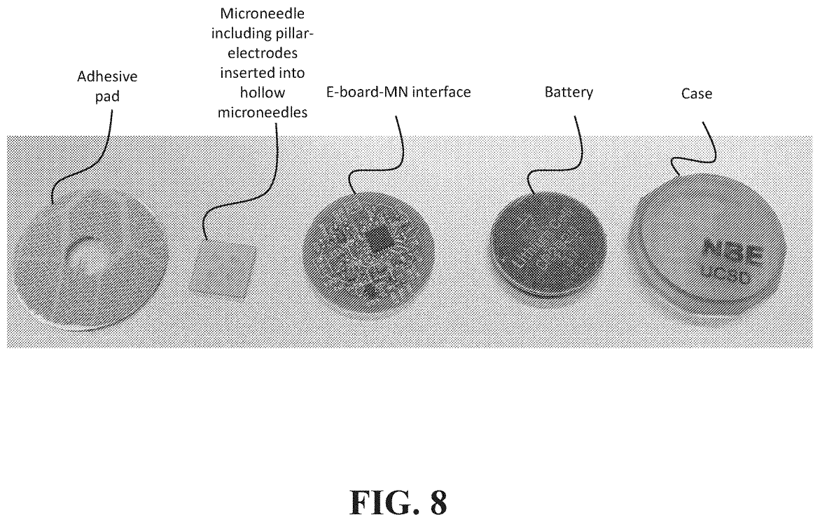

FIG. 8 shows an example of system integration and a microneedle-based electrochemical biosensor board interface. In some implementations, the microneedle-based electrochemical biosensor may include E-board-MN interface, which includes related contacts, microneedles including hollow microneedles and pillar-electrodes inserted into the hollow microneedles, and sealing of the electrodes within the hollow microneedles, for addressing the reproducibility and manual processes. In some implementations, the microneedle-based electrochemical biosensor may also include an adhesive pad, battery, and case. In some implementations, the quick connect E-interface is implemented using spring loaded pogo-pins. In one example, the pins are aligned with conductors on the electrodes and held in place by a 3D printed enclosure to make the electrical connection between the potentiostat and the sensor.

Expansion of the microneedle-based ISF monitoring concept to biomarkers other than glucose is an area of active interest, for minimally-invasive healthcare monitoring in general, and towards diabetes management, in particular. Such a real-time ISF monitoring will depend on developing high performance microneedle sensors that replicate the accuracy and stability of commercial CGM devices. Herein, we have demonstrated the first example of a CKM system based on a microneedle sensor array toward real-time ISF monitoring of ketone bodies. Such monitoring system based on the use of NAD-dependent dehydrogenase enzyme has been realized by addressing the key challenges related to the durable enzyme/cofactor confinement and the stable, low-potential and anti-fouling electrocatalytic system toward NADH oxidation. The resultant microneedle electrode platform displayed very attractive analytical performance in phantom gel skin-mimicking model as well as in artificial ISF medium, with high stability, sensitivity, wide linear range and high selectivity. By integrating the promising performance of the developed CKM system with a GL monitoring approach on the same microneedle array and demonstrating simultaneous HB/GL detection in artificial ISF medium, we presented the first example of a single electrochemical device capable of minimally-invasive ISF monitoring of these key diabetes biomarkers. While these preliminary results offer considerable promise toward the high quality management and treatment of DK/DKA, large scale clinical on-body trials and validation on human subjects are underway for assessing the new device towards the management of diabetes. Ultimately, the new microneedle sensor array strategy can be expanded for the monitoring of other important substrates of dehydrogenase enzymes, and opens new possibilities for multiplexed simultaneous detection of a wide range of analytes.

### Examples

In some embodiments in accordance with the present technology (example 1), an epidermal electrochemical sensor device includes an electrochemical sensor comprising a substrate and an array of microneedles disposed on the substrate, each microneedle having a protruded needle structure and one of a hollow interior inside the protruded needle structure or a coating over at least a portion of the protruded needle structure, wherein the array of microneedles comprises at least one microneedle that includes a working electrode configured within the hollow interior or in the coating to interact with one or more chemical or biological substances that come in contact with the protruded needle structure, and at least one microneedle including a counter electrode configured within the hollow interior or in the coating to measure an electrical potential difference with the working electrode; and a transmitter in communication with the electrochemical sensor to generate output signals based on the electrical potential difference with the working electrode and the counter electrode.

Example 2 includes the sensor device of any of examples 1-15, wherein the output signal is based on a β-hydroxybutyrate dehydrogenase (HBD) enzyme biocatalytic reaction involving the one or more chemical or biological substances and the working electrode.

Example 3 includes the sensor device of any of examples 1-15, wherein the one or more chemical or biological substances include β-hydroxybutyrate (HB), and the output signal is based on HB levels detected by the sensor device based on the HBD enzyme biocatalytic reaction.

Example 4 includes the sensor device of any of examples 1-15, wherein the at least one microneedle including the working electrode includes a carbon paste transducer coating comprising one or more of graphite, carbon nanotubes, or graphene and a pasting liquid including ionic liquid (IL) or mineral oil.

Example 5 includes the sensor device of any of examples 1-15, wherein the at least one microneedle including the working electrode further includes a mediator including one or more of a phenazine, a phenothiazine, a phenoxazine, or a quinoid organic compounds to catalyze oxidation reaction of nicotinamide-adenine dinucleotide (NADH) molecule.

Example 6 includes the sensor device of any of examples 1-15, wherein the mediator is phenanthroline dione (PD), meldola blue, tetracyanoquinodimethane (TCNQ), or tetrathiofulvalene (TTF).

Example 7 includes the sensor device of any of examples 1-15, wherein the pasting liquid includes the ionic liquid (IL) including 1-Ethyl-3-methylimidazolium bis (trifluoromethylsulfonyl) imide (IL).

Example 8 includes the sensor device of any of examples 1-15, wherein the at least one microneedle including the working electrode is covered with an outer polymer layer including at least one polymeric layer.

Example 9 includes the sensor device of any of examples 1-15, wherein the at least one polymeric layer includes at least one of chitosan (Chit), polyvinylchloride (PVC), or a permselective or semipermeable polymeric membrane, including Nafion, polyurethane, cellulose acetate.

Example 10 includes the sensor device of any of examples 1-15, wherein the at least one microneedle including the counter electrode includes carbon paste (CP) or an electrically conducive wire within the hollow interior or a sputtered electrically conductive film on the coating.

Example 11 includes the sensor device of any of examples 1-15, wherein the array of microneedles further includes at least one hollowed microneedle or coated microneedle including a reference electrode to provide control of potential at the at least one microneedle including the working electrode.

Example 12 includes the sensor device of any of examples 1-15, wherein the array of microneedles includes a glucose sensing electrode.

Example 13 includes the sensor device of any of examples 1-15, wherein the array of microneedles includes one or more of a β-hydroxybutyrate microneedle sensing electrode, a glucose microneedle sensing electrode, or a lactate microneedle sensing electrode.

Example 14 includes the sensor device of any of examples 1-15, wherein the at least one microneedle including the working electrode includes multiple functionalization layers including at least one of mediator, enzyme, or cofactor formed on a surface of the microneedle electrode.

Example 15 includes the sensor device of any of examples 1-14, wherein the transmitter includes a wireless transmitter configured to transmit radio frequency signals modulated with the output signal.

In some embodiments in accordance with the present technology (example 16), an epidermal electrochemical sensor device for detection of diabetes biomarkers includes a substrate; a plurality of microneedle electrodes coupled to the substrate and operable to penetrate within skin and contact the microneedle electrodes with interstitial fluid when the device is attached to skin of a user, each microneedle electrode including a microneedle structure and an electrode structure, wherein each microneedle structure includes an exterior wall spanning outward from a base surface of the microneedle structure and forming an apex at a terminus point of the exterior wall, and the electrode structure is configured within a hollow interior region of the microneedle structure or on at least a portion of the exterior wall of the microneedle structure; an enzymatic functionalization layer coupled to the electrode structure of the first microneedle electrode of the plurality of microneedle electrodes operable to detect β-hydroxybutyrate (HB) in the interstitial fluid through an electrochemically-mediated enzymatic reaction, wherein the enzymatic functionalization layer is immobilized to the electrode structure and comprises a β-hydroxybutyrate dehydrogenase (HBD) enzyme and HBD-cofactor that is unbound to the HBD enzyme; and a redox mediator coupled to the electrode structure of the first microneedle electrode to facilitate electron transfer in the electrochemically-mediated enzymatic reaction, wherein the plurality of microneedle electrodes includes a counter electrode or a reference electrode, or both, configured to apply or detect an electrical signal between the counter electrode and/or reference electrode and the first microneedle electrode.

Example 17 includes the sensor device of any of examples 16-44, wherein the redox mediator is integrated in a material of the electrode structure of the first microneedle electrode.

Example 18 includes the sensor device of any of examples 16-44, wherein the electrode structure of the first microneedle electrode includes a carbon paste transducer comprising (i) one or more of graphite, carbon nanotubes, or graphene and (ii) a pasting liquid comprising one or more of ionic liquid (IL) or mineral oil.

Example 19 includes the sensor device of any of examples 16-44, wherein the electrode structure of the first microneedle electrode includes a printable conductive ink, wherein the enzymatic functionalization layer coupled to the electrode structure of the first microneedle electrode includes a printable ink material entrapping the HBD enzyme and the HBD-cofactor within the printable ink material, and wherein the redox mediator is entrapped within one or both of the printable conductive ink and the printable ink material.

Example 20 includes the sensor device of any of examples 16-44, wherein the enzymatic functionalization layer includes a hydrogel coating that entraps the HBD enzyme, the HBD-cofactor, and the redox mediator within a hydrogel material.

Example 21 includes the sensor device of any of examples 16-44, wherein the HBD-cofactor includes nicotinamide adenine dinucleotide (NAD+).

Example 22 includes the sensor device of example 21 and/or any of examples 16-44, wherein the enzymatic functionalization layer is structured to integrate the HBD enzyme and the NAD+ in a compact material such that the sensor device is operable to detect a level of HB in a reagentless electrochemical redox reaction, where HBD-catalyzed oxidation of the HB oxidizes to acetylacetate (AcAc) with a concomitant reduction of the NAD+ to reduce to nicotinamide adenine dinucleotide (NADH), and where the reduced NADH oxidizes back to the NAD+ based on the electron transfer facilitated by the redox mediator to continuously regenerate the HBD-cofactor.

Example 23 includes the sensor device of example 22 and/or any of examples 16-44, wherein the redox mediator comprises one or more of a phenazine, a phenothiazine, a phenoxazine, or a quinoid organic compounds to catalyze an oxidation reaction of a reduced species of the HBD-cofactor that oxidizes to the HBD-cofactor.

Example 24 includes the sensor device of example 23 and/or any of examples 16-44, wherein the redox mediator includes phenanthroline dione (PD), meldola blue, tetracyanoquinodimethane (TCNQ), or tetrathiofulvalene (TTF).

Example 25 includes the sensor device of any of examples 16-44, further comprising an outer layer including at least one polymeric layer.

Example 26 includes the sensor device of example 25 and/or any of examples 16-44, wherein the at least one polymeric layer includes one or more of chitosan, polyvinylchloride (PVC), or a permselective or semipermeable polymeric membrane comprising at least one of Nafion, polyurethane, or cellulose acetate.

Example 27 includes the sensor device of any of examples 16-44, wherein the enzymatic functionalization layer is immobilized to the electrode structure of the first microneedle electrode by a cross-linking agent.

Example 28 includes the sensor device of example 27 and/or any of examples 16-44, wherein the cross-linking agent comprises glutaraldehyde.

Example 29 includes the sensor device of any of examples 16-44, wherein the sensor device is operable to simultaneously monitor multiple diabetes biomarkers, the device further includes a glucose-sensing enzymatic functionalization layer coupled to the electrode structure of a second microneedle electrode of the plurality of microneedle electrodes operable to detect glucose in the interstitial fluid through a glucose-sensing electrochemically-mediated enzymatic reaction, wherein the glucose-sensing enzymatic functionalization layer is immobilized to the electrode structure and comprises a glucose oxidase (GOx) enzyme and a mediator to facilitate electron transfer in a redox reaction.

Example 30 includes the sensor device of example 29 and/or any of examples 16-44, wherein the mediator includes Prussian blue (PB).

Example 31 includes the sensor device of example 29 and/or any of examples 16-44, wherein glucose-sensing enzymatic functionalization layer further includes a permeable polymer film that immobilizes the GOx and the mediator to the electrode structure of the second microneedle electrode.

Example 32 includes the sensor device of example 31 and/or any of examples 16-44, wherein the permeable polymer film comprises a one or more of chitosan, polyvinylchloride (PVC), or a permselective or semipermeable polymeric membrane comprising at least one of Nafion, polyurethane, or cellulose acetate.

Example 33 includes the sensor device of any of examples 16-44, wherein the sensor device is operable to simultaneously monitor multiple diabetes biomarkers, the device further includes a lactate-sensing enzymatic functionalization layer coupled to the electrode structure of a third microneedle electrode of the plurality of microneedle electrodes operable to detect lactate in the interstitial fluid through a lactate-sensing electrochemically-mediated enzymatic reaction, wherein the lactate-sensing enzymatic functionalization layer is immobilized to the electrode structure and comprises a lactate oxidase (LOx) enzyme and a mediator to facilitate electron transfer in a redox reaction.

Example 34 includes the sensor device of example 33 and/or any of examples 16-44, wherein the mediator includes Prussian blue (PB).

Example 35 includes the sensor device of example 33 and/or any of examples 16-44, wherein lactate-sensing enzymatic functionalization layer further includes a permeable polymer film that immobilizes the LOx and the mediator to the electrode structure of the third microneedle electrode.

Example 36 includes the sensor device of example 35 and/or any of examples 16-44, wherein the permeable polymer film comprises a one or more of chitosan, polyvinylchloride (PVC), or a permselective or semipermeable polymeric membrane comprising at least one of Nafion, polyurethane, or cellulose acetate.

Example 37 includes the sensor device of any of examples 16-44, wherein the counter electrode and/or the reference electrode includes carbon paste (CP) or an electrically conducive wire.

Example 38 includes the sensor device of any of examples 16-44, wherein the electrode structure is configured as a coating on at least a portion of the microneedles structure of the first microneedle electrode.

Example 39 includes the sensor device of any of examples 16-44, wherein, at a portion of the exterior wall, the microneedle structure of the first microneedle electrode has an opening leading in to the hollow interior region of the microneedle structure that is defined by an interior wall, wherein the electrode structure is at least partially contained within the hollow region.

Example 40 includes the sensor device of any of examples 16-44, wherein the microneedle structure of each of the plurality of microneedle electrodes includes a pyramidal geometry, a conical geometry, or a combination thereof.

Example 41 includes the sensor device of any of examples 16-44, further including a plurality of electrical conduits, each coupled to the electrode structure of each of the microneedle electrodes and disposed on or within the substrate, wherein each electrical conduit terminates at an interface portion of the electrical conduit.

Example 42 includes the sensor device of any of examples 16-44, further including an electrical circuit electrically connected to the plurality of electrical conduits to process the electrical signal as a processed signal.

Example 43 includes the sensor device of example 42 and/or any of examples 16-44, including a wireless transmitter in communication with the electrical circuit to transmit the processed signal.

Example 44 includes the sensor device of example 42 or example 43 and/or any of examples 16-41, comprising a data processing unit in communication with the electrical circuit via electrical connection or wireless communication to process the processed signal and determine a level of the detected HB.

In some embodiments in accordance with the present technology (example 45), a sensor device includes a plurality of protruded working needle structures including a first working electrode structured to perform a first electrochemical detection of a first chemical or biological substance and a second working electrode structured to perform a second electrochemical detection of a second chemical or biological substance; and a plurality of protruded functional needle structures including a reference electrode structured to provide a reference electrical potential to the plurality of protruded working needle structures and a counter electrode structured to measure an electrical potential difference between the reference electrical potential and the protruded working needle structures, wherein the first and second working electrodes are structured to share the counter electrode and the reference electrode to detect the first and second chemical or biological substance simultaneously.

Example 46 includes the sensor device of any of examples 45-62, wherein at least one of the first and second working electrodes includes a carbon paste transducer coating comprising one or more of graphite, carbon nanotubes, or graphene and a pasting liquid including ionic liquid (IL) or mineral oil.

Example 47 includes the sensor device of any of examples 45-62, wherein the at least one of the first and second working electrodes further includes a mediator including one or more of a phenazine, a phenothiazine, a phenoxazine, or a quinoid organic compounds to catalyze oxidation reaction of nicotinamide-adenine dinucleotide (NADH) molecule.

Example 48 includes the sensor device of any of examples 45-62, wherein the mediator is phenanthroline dione (PD), meldola blue, tetracyanoquinodimethane (TCNQ), or tetrathiofulvalene (TTF).

Example 49 includes the sensor device of any of examples 45-62, wherein the pasting liquid includes the ionic liquid (IL) including 1-Ethyl-3-methylimidazolium bis (trifluoromethylsulfonyl) imide (IL).

Example 50 includes the sensor device of any of examples 45-62, wherein at least one of the first and second working electrodes is covered with an outer polymer layer including at least one polymeric layer.

Example 51 includes the sensor device of any of examples 45-62, wherein the at least one polymeric layer includes at least one of chitosan, polyvinylchloride, or a permselective or semipermeable polymeric membrane, including Nafion, polyurethane, cellulose acetate.

Example 52 includes the sensor device of any of examples 45-62, wherein the first chemical or biological substance includes ketone bodies.

Example 53 includes the sensor device of any of examples 45-62, wherein the ketone bodies include a β-hydroxybutyrate (HB).

Example 54 includes the sensor device of any of examples 45-62, wherein the first electrochemical detection includes a bio-electrocatalytic process for detecting the (β-hydroxybutyrate (HB) based on a nicotinamide adenine dinucleotide (NAD) dependent dehydrogenase enzyme.

Example 55 includes the sensor device of any of examples 45-62, wherein the first electrochemical detection includes a β-hydroxybutyrate dehydrogenase (HBD) enzyme-catalyzed oxidation of the β-hydroxybutyrate (HB) to acetylacetate (AcAc).

Example 56 includes the sensor device of any of examples 45-62, wherein the first electrochemical detection further includes a concomitant reduction of cofactor an oxidized form of nicotinamide adenine dinucleotide (NAD+) to a reduced form of nicotinamide adenine dinucleotide (NADH)

Example 57 includes the sensor device of any of examples 45-62, wherein the second chemical or biological substance includes glucose.

Example 58 includes the sensor device of any of examples 45-62, wherein the second electrochemical detection includes an oxidation from glucose to gluconate.

Example 59 includes the sensor device of any of examples 45-62, wherein the oxidation includes an enzymatic glucose oxidase (GOx) catalyzed oxidation.

Example 60 includes the sensor device of any of examples 45-62, wherein the second electrochemical detection includes a reduction of (H₂O₂) enzymatic product of the oxidation of the glucose.

Example 61 includes the sensor device of any of examples 45-62, wherein the counter electrode includes at least one of graphite powder (GP) or mineral oil (MO) paste.

Example 62 includes the sensor device of any of examples 45-61, wherein the reference electrode includes at least one of Ag or AgCl.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1-15. (canceled)

16. An epidermal electrochemical sensor device for detection of diabetes biomarkers, comprising:
a substrate;
a plurality of microneedle electrodes coupled to the substrate and operable to penetrate within skin and contact the microneedle electrodes with interstitial fluid when the device is attached to skin of a user, each microneedle electrode including a microneedle structure and an electrode structure, wherein each microneedle structure includes an exterior wall spanning outward from a base surface of the microneedle structure and forming an apex at a terminus point of the exterior wall, and the electrode structure is configured within a hollow interior region of the microneedle structure or on at least a portion of the exterior wall of the microneedle structure;
an enzymatic functionalization layer coupled to the electrode structure of the first microneedle electrode of the plurality of microneedle electrodes operable to detect β-hydroxybutyrate (HB) in the interstitial fluid through an electrochemically-mediated enzymatic reaction, wherein the enzymatic functionalization layer is immobilized to the electrode structure and comprises a β-hydroxybutyrate dehydrogenase (HBD) enzyme and HBD-cofactor that is unbound to the HBD enzyme; and
a redox mediator coupled to the electrode structure of the first microneedle electrode to facilitate electron transfer in the electrochemically-mediated enzymatic reaction,
wherein the plurality of microneedle electrodes includes a counter electrode or a reference electrode, or both, configured to apply or detect an electrical signal between the counter electrode and/or reference electrode and the first microneedle electrode.

17. The sensor device of claim 16, wherein the redox mediator is integrated in a material of the electrode structure of the first microneedle electrode.

18. The sensor device of claim 16, wherein the electrode structure of the first microneedle electrode includes a carbon paste transducer comprising (i) one or more of graphite, carbon nanotubes, or graphene and (ii) a pasting liquid comprising one or more of ionic liquid (IL) or mineral oil.

19. The sensor device of claim 16, wherein the electrode structure of the first microneedle electrode includes a printable conductive ink, wherein the enzymatic functionalization layer coupled to the electrode structure of the first microneedle electrode includes a printable ink material entrapping the HBD enzyme and the HBD-cofactor within the printable ink material, and wherein the redox mediator is entrapped within one or both of the printable conductive ink and the printable ink material.

20. The sensor device of claim 16, wherein the enzymatic functionalization layer includes a hydrogel coating that entraps the HBD enzyme, the HBD-cofactor, and the redox mediator within a hydrogel material.

21. The sensor device of claim 16, wherein the HBD-cofactor includes nicotinamide adenine dinucleotide (NAD+).

22-24. (canceled)

25. The sensor device of claim 16, further comprising an outer layer including at least one polymeric layer.

26. (canceled)

27. The sensor device of claim 16, wherein the enzymatic functionalization layer is immobilized to the electrode structure of the first microneedle electrode by a cross-linking agent.

28. The sensor device of claim 27, wherein the cross-linking agent comprises glutaraldehyde.

29. The sensor device of claim 16, wherein the sensor device is operable to simultaneously monitor multiple diabetes biomarkers, the device further comprising:
a glucose-sensing enzymatic functionalization layer coupled to the electrode structure of a second microneedle electrode of the plurality of microneedle electrodes operable to detect glucose in the interstitial fluid through a glucose-sensing electrochemically-mediated enzymatic reaction, wherein the glucose-sensing enzymatic functionalization layer is immobilized to the electrode structure and comprises a glucose oxidase (GOx) enzyme and a mediator to facilitate electron transfer in a redox reaction.

30. (canceled)

31. The sensor device of claim 29, wherein the glucose-sensing enzymatic functionalization layer further includes a permeable polymer film that immobilizes the GOx and the mediator to the electrode structure of the second microneedle electrode.

32. (canceled)

33. The sensor device of claim 16, wherein the sensor device is operable to simultaneously monitor multiple diabetes biomarkers, the device further comprising:
a lactate-sensing enzymatic functionalization layer coupled to the electrode structure of a third microneedle electrode of the plurality of microneedle electrodes operable to detect lactate in the interstitial fluid through a lactate-sensing electrochemically-mediated enzymatic reaction, wherein the lactate-sensing enzymatic functionalization layer is immobilized to the electrode structure and comprises a lactate oxidase (LOx) enzyme and a mediator to facilitate electron transfer in a redox reaction.

34. (canceled)

35. The sensor device of claim 33, wherein the lactate-sensing enzymatic functionalization layer further includes a permeable polymer film that immobilizes the LOx and the mediator to the electrode structure of the third microneedle electrode.

36. (canceled)

37. The sensor device of claim 16, wherein the counter electrode and/or the reference electrode includes carbon paste (CP) or an electrically conducive wire.

38. The sensor device of claim 16, wherein the electrode structure is configured as a coating on at least a portion of the microneedles structure of the first microneedle electrode.

39. The sensor device of claim 16, wherein, at a portion of the exterior wall, the microneedle structure of the first microneedle electrode has an opening leading in to the hollow interior region of the microneedle structure that is defined by an interior wall, wherein the electrode structure is at least partially contained within the hollow region.

40. The sensor device of claim 16, wherein the microneedle structure of each of the plurality of microneedle electrodes includes a pyramidal geometry, a conical geometry, or a combination thereof.

41. The sensor device of claim 16, further comprising:
a plurality of electrical conduits, each coupled to the electrode structure of each of the microneedle electrodes and disposed on or within the substrate, wherein each electrical conduit terminates at an interface portion of the electrical conduit.

42. The sensor device of claim 41, further comprising:
an electrical circuit electrically connected to the plurality of electrical conduits to process the electrical signal as a processed signal.

43. The sensor device of claim 42, further comprising:
a wireless transmitter in communication with the electrical circuit to transmit the processed signal.

44. (canceled)
